(19) United States
(12) Patent Application Publication
(10) Pub. No.: US 2026/0018242 A1
(43) Pub. Date: Jan. 15, 2026

(54) GUIDED POSITIONAL SCANNING METHOD

(71) Applicant: Immatics Biotechnologies GmbH, Tuebingen (DE)

(72) Inventors: Michael ROEMER, Tuebingen (DE); Jens FRITSCHE, Tuebingen (DE); Heiko SCHUSTER, Tuebingen (DE); Dominik MAURER, Tuebingen (DE); Claudia WAGNER, Tuebingen (DE); Alena BUSCHE, Tuebingen (DE)

(21) Appl. No.: 18/994,259

(22) PCT Filed: Aug. 7, 2023
(86) PCT No.: PCT/EP2023/071872

§ 371 Date: Jan. 14, 2025

Related U.S. Application Data
(60) Provisional application No. 63/395,934, filed on Aug. 8, 2022.

(30) Foreign Application Priority Data
Aug. 8, 2022 (EP) 22189353.0

(51) Int. Cl. G16B 15/30; G01N 33/68; G16B 15/20; G16B 40/20

(52) U.S. Cl. CPC G16B 15/30; G01N 33/6845; G16B 15/20; G16B 40/20

(57) ABSTRACT

The present invention relates to methods for identifying alternative target peptides of a binding moiety.

The present invention relates to methods for identifying alternative target peptides of a binding moiety.

### BACKGROUND OF THE INVENTION

The major histocompatibility complex (MHC) is an essential component of the adaptive immune system responsible for antigen presentation at the cell surface. In a cell, proteins are constantly synthesized and proteosomally degraded. Short peptide fragments of these degraded proteins are presented by the MHC molecules on the cell surface. In case a cell suffers from viral infection, intracellular microorganism infection, or cancerous transformation, such proteins degraded in the proteosome are as well loaded onto MHC molecules and presented on the cell surface. This enables T lymphocytes to detect these MHC presented peptides with their T cell receptor (TCR) and initiate immune reactions in case of non-self-antigens being presented.

This mechanism enables the use of binding moieties able to specifically bind to MHC presented peptides, such as TCRs, antibodies and antigen binding fragments thereof, as therapeutics in the treatment of various diseases, including cancer. The use of such binding moieties has the potential to overcome the shortcomings of common antibody-based therapeutics, which naturally bind only antigens expressed on the cell surface. However, by targeting also intracellular protein, which is in the context of cancer a very relevant proportion of cancer antigens, this allows new ways of treating cancer and other diseases.

It is however essential that such therapeutic binding moieties directed against proteolyzed intra- and extracellular peptides presented in the context of the MHC are very specific with regard to their MHC presented target peptide and should not cross react with other target peptides accessible in a subject to be treated. It is therefore necessary to identify if such binding moieties can also bind to other MHC-presented target peptides than those they are supposed to bind.

In the prior art a mutational scan based on the amino acid sequence of the target peptide has been proposed to identify alternative target peptides. These mutational scans were originally based on the substitution of each amino acid with a specific amino acid such as alanine or glycine (i.e. alanine- or glycine scan) to identify critical sites for binding to the target peptide. The use of specific amino acids such as alanine or glycine on each position of the target peptide is suitable to identify amino acids critical for binding but does not allow to observe the effect the substitution with other amino acids might have. Therefore full mutational scans were proposed that use all 19 alternative naturally-occurring amino acids on each position of the target peptide (see e.g. Maier et al. 2000 (Eur. J. Immunol., 30: 448-457), Harper et al. (2018), PLoS ONE 13(10):e0205491; WO2014/096803A1). While this allows insights into the effect of other amino acids in the target peptide this also vastly increases the amount of alternative peptides to be synthesized and tested.

In an attempt to address this drawback it was proposed to exclude specific amino acid positions (e.g. the anchor positions) from a full mutational scan to reduce the amount of testing required (discontinuous mutational scan; Karapetyan et al (2019), Front. Immunol. 10:2501.doi: 10.3389/fimmu.2019.02501). While reducing the amount of testing required this also affects the specificity and sensitivity of such a method with regard to identifying alternative targets of a binding moiety.

The present invention discloses a reduced mutational scan that does not require to perform amino acid exchanges at every position of a target peptide but rather uses a replacement matrix to specify amino acid positions to be mutated. Furthermore a method for scoring and ranking alternative target peptides based on parameters obtained in a mutational scan is provided in the present invention. Finally a method, which allows to identify alternative target peptides without any additional experimentation that can be combined with the scoring and ranking method is disclosed herein.

### SUMMARY OF THE INVENTION

In a first aspect, the present invention provides a method for the identification of alternative target peptides of a binding moiety comprising the steps of:
a) providing a target peptide of the binding moiety;
b) generation of a replacement matrix for the target peptide, wherein the replacement matrix provides a selection of alternative amino acids for amino acids of the target peptide,
   with the proviso that
      the replacement matrix contains less alternative amino acids than the number of all possible alternative amino acids and the selection of alternative amino acids is not identical in all selections;
c) generating peptide variants of the target peptide according to the replacement matrix of step b), wherein each peptide variant comprises only one alternative amino acid as compared to the target peptide; determining at least one binding parameter of the peptide variants to the binding moiety;
d) providing a list of potential alternative target peptides of the binding moiety comprising the following steps:
   i) calculating a position-specific binding value for the alternative amino acid present in the peptide variant of step c), based on the at least one binding parameter of the peptide variant determined in step c),
   ii) providing a list of amino acid sequences having a specified length and being comprised in a protein database, preferably a proteome or ligandome database,
   iii) assigning an alternative target peptide value to each amino acid sequence of step ii) based on the position-specific binding values for each alternative amino acid present in the amino acid sequence;
   iv) selecting the potential alternative target peptides from the list of amino acid sequences of step ii) based on the binding peptide values assigned in step iii);
e) determining at least one binding parameter of the potential alternative target peptides to the binding moiety, wherein alternative target peptides are identified based on at least one binding parameter.

In a second aspect the present invention provides a method for the identification of alternative target peptides of a binding moiety based on a substitution analysis of a target peptide of said binding moiety, comprising the following steps:
I) determining a position-specific binding value for each alternative amino acid in the substitution analysis of the target peptide, based on at least one binding parameter determined in the mutational scan;
II) providing a list of amino acid sequences having a specified length and being comprised in a protein database, preferably a proteome or ligandome database;
III) assigning an alternative target peptide value to each amino acid sequence of step II) based on the position-specific binding values of each alternative amino acid of step I) present in the amino acid sequence;
IV) selecting the potential alternative target peptides from the list of amino acid sequences of step II) based on the alternative target peptide values assigned in step III);
V) determining at least one binding parameter of the potential alternative target peptides to the binding moiety, wherein alternative target peptides are identified based on at least one binding parameter.

### LIST OF FIGURES

In the following, the content of the FIGS. comprised in this specification is described. In this context please also refer to the detailed description of the invention above and/or below.

FIG. 1 refers to a comparative example of a replacement matrix using a full mutational scan (i.e. full scan as disclosed in Maier et al. (2000), Harper et al. (2018) and WO2014/096803A1), a partial scan (according to the method disclosed in Karapetyan et al. 2019, Front. Immunol. 10:2501.doi: 10.3389/fimmu.2019.02501) and the replacement matrix of the present invention (XPRES-Scan). The replacement matrix depicts on the left side 20 amino acids in one-letter code used in the mutational scan as replacements. On the top side the position within the 9-mer target peptide is indicated with the anchor positions being underlined. The amino acid sequence of the target peptide is indicated by the grey filled circles (i.e. ‘target’). The unfilled circles indicate positions of amino acid exchange (i.e. ‘exchange’) and the black filled circles indicate positions excluded from the mutational scan (i.e. ‘no exchange’). The partial-scan and the XPRES-Scan of the present invention significantly reduce the number of amino acid exchanges required for the mutational scan. The replacement matrix used for XPRES-Scan in this example is specific for HLA (A*02:01) and uses a position specific amino acid frequency of 0.5%. Using a higher frequency as cut off would result in an even lower number of amino acid exchanges.

FIG. 2: refers to an example that depicts the position specific amino acid frequency in peptides presented by the target HLA (see example 2). Based on the position specific frequency a replacement matrix can be generated by applying the desired cut off (e.g. at least 2%, at least 1.5%, at least 1%, at least 0.5%, etc). Only positions in which the threshold is reached are then to be included in the replacement matrix. The present example indicates a cut-off of at least 1% by the filled boxes.

FIG. 3: refers to the results of a mutational scan depicting the binding values determined for the tested peptide variants of the target peptide. Positions in the replacement matrix that were, due to the position specific amino frequency (see FIG. 2), not included in the mutational scan are marked as ‘#N/A’.

FIG. 4: refers to the interpolation of missing values in the mutational scan as explained in example 2 (see 2.1.4) in more detail. In brief for positions not included in the mutational scan (i.e. #N/A in FIG. 3) the median of the binding value of the measured mutational variants for each non-anchor position (1,3,4,5,6,7,8) was imputed.

FIG. 5: refers to the binding scores of each individual position and the binding score calculation for the target peptide variants. The calculation is described in more detail in example 2 (see section 2.1.6).

FIG. 6: refers to the precision-recall-curves (PR curve or PRC) that depicts the (TP precision

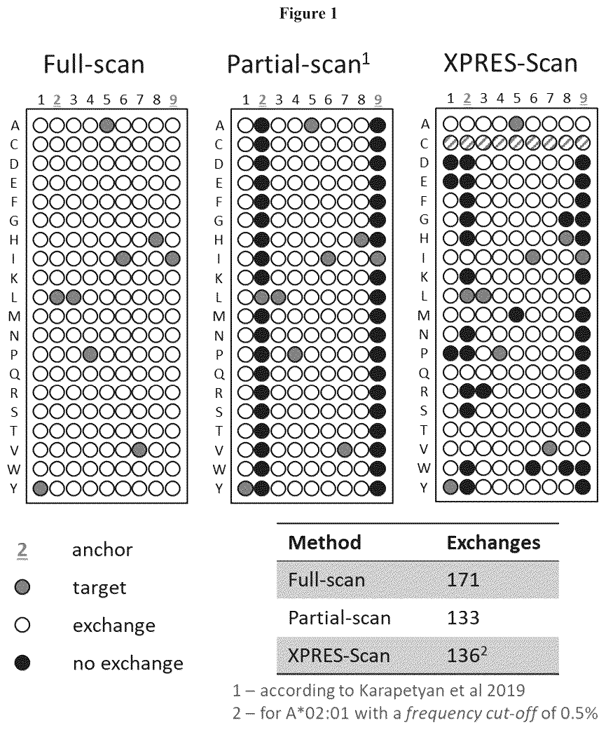

$$( \frac{TP}{TP + FP} ,$$

where TP is the number of true positives, i.e., predicted alternative target peptides that are confirmed alternative target peptides, and FP is the number of false positives, i.e., predicted alternative target peptides that are not confirmed alternative target peptides) and the recall

$$( \frac{TP}{TP + FN} ,$$

where TP is defined as above and FN is the number of false negatives, i.e., confirmed alternative target peptides that are not predicted alternative target peptides) for each possible cut-off, i.e., the peptides are sorted by their predicted binding score and precision and recall are calculated for each unique binding score and shown in the graph. The area under the PRC (PRC-AUC) can be used as a measure to compare different methods globally (i.e. without looking at individual cut-offs for each method), where a higher PRC-AUC indicates a better performance. The methods shown in this figure are the XPRES-Scan as described in example 2 (see section 2.1), the full-scan (example 2, section 2.2) and the partial-scan (Example 2, section 2.3). The AUC in the legend refers to the PRC-AUC for each of the method.

FIG. 7: refers to the precision-recall-curves of models described in examples 2, 4 and 5 when used to identify alternative target peptides in the UniProt protein database (datasets “Reviewed (Swiss-Prot)” and “Isoform sequences”, release version 2020_06). The evaluated methods are the XPRES-Scan (example 2, section 2.1) with different interpolation functions (“LOD” refers to interpolation with limit of detection, “median” refers to interpolation with the median of measured amino acids at the position, “min” refers to interpolation with the minimum of measured amino acids at the position) and amino-acid frequency thresholds (at least 1% or at least 0.5% position-specific amino-acid frequency in the dataset by Abelin et al., 2017), the full-scan (see example 2, 2.2), the partial-scan (see example 2, 2.3), the PSSM-based alternative target peptide prediction as described in example 4 using either the PMBEC amino-acid similarity matrix (“PMBEC”), the BLOSUM62 amino-acid similarity matrix (“BLOSUM”), the full-scan-derived similarity matrix described in example 2 (“full-scan (logsum)”, section 2.2), or the partial-scan-derived similarity matrix described in example 2 (“partial-scan (logsum)”, section 2.3), and the combination of the PSSM-based alternative target peptide prediction with a single-scan as described in example 5 using a single-scan combined with the PMBEC amino-acid similarity matrix (“PMBEC+Single-scan”) or the BLOSUM62 amino-acid similarity matrix (“BLOSUM+Single-scan”).

FIG. 8: refers to the precision-recall-curves of models described in examples 2, 4 and 5 when used to identify alternative target peptides in the UniProt protein database (datasets “Reviewed (Swiss-Prot)” and “Isoform sequences”, release version 2020_06) after the 9-mers were filtered with NetMHC 3.0 (predicted HLLA A*02:01 affinity≤500 nM) following the procedure disclosed in Karapetyan et al. The evaluated methods are the same as in FIG. 7.

FIG. 9: refers to the precision-recall-curves of models described in examples 2, 4 and 5 when used to identify alternative target peptides in the XPRESIDENT® ligandome database. The evaluated methods are the same as in FIG. 7.

FIG. 10: refers to the precision-recall-curves of models described in example 2 when used to identify alternative target peptides in the XPRESIDENT® ligandome database. The evaluated methods are the full-scan (see example 2, 2.2), the partial-scan (see example 2, 2.3) using the XPRESIDENT® ligandome database. Furthermore a comparison of full-scan using the procedure disclosed in Maier et al. 2000 and Harper et al. ((2018) (i.e. ‘full-scan’) and partial-scan using the procedure disclosed in Karapetyan et al (i.e. ‘partial-scan’) is compared with a full-scan and partial-scan using the method of the second aspect of the invention (i.e. ‘full-scan (logsum)’ and ‘partial-scan (logsum)’).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

The term “substitution analysis” according to the invention relates to any method in which the contribution of individual amino acids of a peptide to a functional parameter or property of the peptide is determined. Examples of substitution analysis are mutational scans in which a single amino acid of a peptide are replaced by at least one other amino acid and a binding parameter of the resulting variant peptide is determined. Examples known in the art of such mutational scans include a full mutational scan (referred to herein as ‘full-scan’) in which every amino acid of a peptide is replaced with any of the other proteinogenic amino acids and the resulting peptide variants having one modified (‘mutated’) amino acid each are then used to determine the functional parameter of interest (preferably binding parameters). Other examples of such mutational scans known in the art replacing the amino acids of the peptide with only a single amino acid such as Ala or Gly are also known in the art (referred to herein as ‘single-scan’). Any of these substitution analysis can be combined with the method of the second aspect of the invention. The generation of the replacement matrix and the peptide variants according to the first aspect of the invention (i.e. steps b) and c)) is a preferred example of a substitution analysis. In a preferred embodiment the substitution analysis does not require the generation of peptide variants (i.e. no laboratory work is required), but instead determines the contribution of individual amino acids of a peptide to a functional parameter of the peptide based on amino acid similarity measure. Non-limiting examples of such amino acid similarity measures are PMBEC (see Kim et al; BMV Bioinformatics 2009; 10:394) and evolutionary relations between amino acids (e.g. BLOSUM; see Henikoff & Henikoff; PNAS 1992 Nov. 15; 89(22): 10915-10919)). In another preferred embodiment the substitution analysis combines the use of amino acid similarity measures with a single amino acid mutational scan (i.e. a single-scan). A preferred example of a single scan is using alanine or glycine (preferably alanine).

The term “binding moiety” as used herein, refers to molecules that contain an antigen-binding site that specifically binds an antigen. Comprised in this term are immunoglobulin and immunoglobulin-like proteins that specifically bind to a target peptide or target epitope. Preferred examples of binding moieties are T cell receptors (TCRs), antibodies and antigen-binding fragments thereof, preferably TCRs and antigen binding fragments thereof. This term also includes binding moieties with more than one binding site (i.e. multispecific binding moieties), preferably bispecific binding moieties. A preferred example of a bispecific binding moiety includes the binding site of a TCR and the binding site of an antibody.

A “T-cell receptor (TCR)” in the context of the present invention is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβ TCR and γδ TCR each contain two polypeptides, each of which has a membrane-proximal constant domain, and a membrane-distal variable domain. Each of the constant and variable domains include an intra-chain disulfide bond. The variable domains contain the highly polymorphic loops analogous to the complementarity determining regions (CDRs) of antibodies.

The term “TCR” herein denotes TCRs and fragments thereof, as well as single chain TCRs and fragments thereof, in particular variable alpha and beta domains of single domain TCRs, and chimeric, humanized, bispecific or multispecific TCRs.

“Fragments of a TCR” comprise a portion of an intact or native TCR, in particular the antigen binding region or variable region of the intact or native TCR. Examples of TCR fragments include fragments of the α, β, δ, γ chain, such as Vα-Ca or V3-Cβ or portions thereof, such fragments might also further comprise the corresponding hinge region or single variable domains, such as Vα, Vβ, Vδ, Vγ, single chain VαVβ fragments or bispecific and multispecific TCRs formed from TCR fragments. Fragments of a TCR exert identical functions compared to the naturally occurring full-length TCR, i.e. fragments selectively and specifically bind to their target peptide.

In an “antibody” two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site (synonym to antibody binding site) and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

In the context of the invention, the antibody is an IgM, IgD, IgG, IgA or IgE, preferably IgG.

“Antibody Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. Accordingly, the light chain variable domain may thus be designated as (FR1-L)-(CDR1-L)-(FR2-L)-(CDR2-L)-(FR3-L)-(CDR3-L)-(FR4-L) and the heavy chain variable domain may thus be designated as (FR1-H)-(CDR1-H)-(FR2-H)-(CDR2-H)-(FR3-H)-(CDR3-H)-(FR4-H).

In the context of the invention, CDR/FR definition in an immunoglobulin light or heavy chain is to be determined based on Kabat numbering (Kabat E A, Te, Wu T, Foeller C, Perry H M, Gottesman K S. (1992) Sequences of Proteins of Immunological Interest.).

The term “antibody” denotes antibodies and fragments thereof, as well as single domain antibodies and fragments thereof, in particular a variable heavy chain of a single domain antibody, and chimeric, humanized, bispecific or multispecific antibodies.

The “major histocompatibility complex” (MHC) in the context of the present invention is a set of cell surface proteins essential for the acquired immune system to recognize foreign molecules in vertebrates, which in turn determines histocompatibility. The main function of MHC molecules is to bind to antigens derived from pathogens and display them on the cell surface for recognition by the appropriate T cells. The human MHC is also called the HLA (human leukocyte antigen) complex (often just the HLA). The MHC gene family is divided into three subgroups: class I, class II, and class III. Complexes of peptide and MHC class I are recognized by CD8-positive T cells bearing the appropriate T cell receptor (TCR), whereas complexes of peptide and MHC class II molecules are recognized by CD4-positive-helper-T cells bearing the appropriate TCR. Since both types of response, CD8 and CD4 dependent, contribute jointly and synergistically to the anti-tumor effect, the identification and characterization of tumor-associated antigens and corresponding T cell receptors is important in the development of cancer immunotherapies such as vaccines and cell therapies. The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface. Each individual can express up to two types of HLA-A, one from each of their parents. Some individuals will inherit the same HLA-A from both parents, decreasing their individual HLA diversity; however, the majority of individuals will receive two different copies of HLA-A. This same pattern follows for all HLA groups. In other words, every single person can only express either one or two of the 2432 known HLA-A alleles.

The MHC class I HLA protein in the context of the present invention may be an HLA-A, HLA-B or HLA-C protein, preferably HLA-A protein, more preferably HLA-A*02.

“HLA-A*02” signifies a specific HLA allele, wherein the letter A signifies the gene and the suffix “*02” indicates the A2 serotype.

In the MHC class I dependent immune reaction, peptides not only have to be able to bind to certain MHC class I molecules expressed by tumor cells, they subsequently also have to be recognized by T cells bearing specific T cell receptors (TCR).

A “MHC-associated peptide epitope” in the context of the present invention is thus an epitope on a peptide that is presented by a MHC molecule and that can be bound by a binding moiety (in particular a TCR or an antibody). Preferably a MHC class I associated peptide has a length of 8 to 11 amino acids, preferably 9 to 10, most preferably 9 amino acids. Preferably a MHC class II associated peptide has a length of 13 to 25 amino acids.

The term “anchor position” according to the invention relates to specific residues in the peptides bound and presented by MHC molecules. The amino acids at the anchor position of the peptide have amino acid side chains that bind into pockets lining the peptide-binding groove of the MHC class I molecule. Each MHC class I molecule binds different patterns of anchor residues, called anchor motifs, giving some specificity to peptide binding. Anchor positions exist but are less obvious for peptides that bind to MHC class II molecules. Methods to identify anchor positions are well known in the art and are described for example in Rammensee et al,; Immunogenetics (1995):178-228).

The term “binding” according to the invention preferably relates to a specific binding. The term “binding affinity” generally refers to the strength of the sum total of noncovalent interactions between a single binding site of a molecule (e.g., an antibody) and its binding partner (e.g., target or antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). “Specific binding” means that a binding moiety (e.g. an antibody) binds stronger to a target such as an epitope for which it is specific compared to the binding to another target. A binding moiety binds stronger to a first target compared to a second target if it binds to the first target with a dissociation constant (K_{d}) which is lower than the dissociation constant for the second target. The dissociation constant (K_{d}) for the target to which the binding moiety binds specifically is more than 10-fold, preferably more than 20-fold, more preferably more than 50-fold, even more preferably more than 100-fold, 200-fold, 500-fold or 1000-fold lower than the dissociation constant (K_{d}) for the target to which the binding moiety does not bind specifically.

Accordingly, the term “K_{d}” (measured in “mol/L”, sometimes abbreviated as “M”) is intended to refer to the dissociation equilibrium constant of the particular interaction between a binding moiety (e.g. a TCR, an antibody or fragment thereof) and a target molecule (e.g. a target peptide or epitope thereof). Affinity can be measured by common methods known in the art, including but not limited to surface plasmon resonance based assay (such as the BIAcore assay); quartz crystal microbalance assays (such as Attana assay); enzyme-linked immunoabsorbent assay (ELISA); and competition assays (e.g. RIA's). Low-affinity binding moieties generally bind antigen slowly and tend to dissociate readily, whereas high-affinity binding moieties generally bind antigen faster and tend to remain bound longer. A variety of methods of measuring binding affinity are known in the art, any of which can be used for purposes of the present invention.

Typically, binding moieties according to the invention bind with a sufficient binding affinity to their target, for example, with a Kd value of between 500 nM-1 pM, i.e. 500 nM, 450 nM, 400 nM, 350 nM, 300 nM, 250 nM, 200 nM, 150 nM, 100 nM, 50 nM, 10 nM, 1 nM, 900 pM, 800 pM, 700 pM, 600 pM, 500 pM, 400 pM, 300 pM, 200 pM, 100 pM, 50 pM, 1 pM.

The term “proteinogenic amino acids” according to the invention relates to amino acids that are incorporated into proteins during translation. The standard genetic code encodes 20 proteinogenic amino acids, i.e. alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y).

### Embodiments

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### First Aspect of the Invention

In a first aspect, the present invention provides a method for the identification of alternative target peptides of a binding moiety comprising the steps of:
a) providing a target peptide of the binding moiety;
b) generation of a replacement matrix for the target peptide, wherein the replacement matrix provides a selection of alternative amino acids for amino acids of the target peptide,
   with the proviso that
      the replacement matrix contains less alternative amino acids than the number of all possible alternative amino acids and
      the selection of alternative amino acids is not identical in all selections;
c) generating peptide variants of the target peptide according to the replacement matrix of step b), wherein each peptide variant comprises only one alternative amino acid as compared to the target peptide; determining at least one binding parameter of the peptide variants to the binding moiety;
d) providing a list of potential alternative target peptides of the binding moiety comprising the following steps:
   i) calculating a position-specific binding value for the alternative amino acid present in the peptide variant of step c), based on the at least one binding parameter of the peptide variant determined in step c),
   ii) providing a list of amino acid sequences having a specified length and being comprised in a protein database, preferably a proteome or ligandome database,
   iii) assigning an alternative target peptide value to each amino acid sequence of step ii) based on the position-specific binding values for each alternative amino acid present in the amino acid sequence;
   iv) selecting the potential alternative target peptides from the list of amino acid sequences of step ii) based on the binding peptide values assigned in step iii);
e) determining at least one binding parameter of the potential alternative target peptides to the binding moiety, wherein alternative target peptides are identified based on at least one binding parameter.

In other words, the method according to the first aspect of the present invention intends to identify additional target peptides that a particular binding moiety can bind to. This method is primarily to be seen in the context of binding moieties that can be used in therapeutic applications. The presence of target peptides other than the intended target peptide can have potentially serious side effects for the therapeutic use of a binding moiety. It is therefore important to identify other peptides (i.e. alternative target peptides) that are cross reactive with the binding moiety. Such alternative target peptides are sometimes also referred to as off-targets.

Target Peptide and Binding Moiety (i.e. Step a)

The target peptide in the context of the present invention refers to a peptide known to be specifically bound by the binding moiety of the present invention if presented as a MHC/HLA associated peptide.

The amino acid sequence of the known target peptide is also the basis for the method of the present invention to identify alternative target peptides that differ from the target peptide with regard to the amino acid sequence.

The target peptide can have different lengths, which primarily depends on the MHC/HLA class the target peptide is presented by. In a preferred embodiment the target peptide is a MHC/HLA class I presented peptide having a length of 8 to 11 amino acids, preferably 9 to 10, most preferably 9 amino acids. In another preferred embodiment the target peptide is a MHC/HLA class II presented peptide having a length of 13 to 25 amino acids.

In a preferred embodiment the target peptide is an 8- to 25-mer, preferably an 8- to 12-mer, more preferably a 9-10-mer, most preferably a 9-mer.

In a preferred embodiment the target peptide is expressed in a disease relevant tissue. Preferred examples of relevant diseases are cancer, auto-immune and infectious diseases, most preferably cancer. In a preferred embodiment the target peptide is expressed specifically in cancer cells (i.e. is tumor-associated).

In a preferred embodiment, the binding moiety of the present invention comprises an antigen binding site selected from T cell receptors (TCRs) and fragments thereof; antibodies and antigen-binding fragments thereof. In a preferred embodiment, the binding moiety comprises or consists of the antigen binding site of a TCR. In a preferred embodiment, the binding moiety comprises or consists of the antigen binding site of an antibody.

In a preferred embodiment, the binding moiety is a bispecific binding moiety comprising antigen binding sites selected from T cell receptors (TCRs) and antigen binding fragments thereof; antibodies and antigen-binding fragments thereof. In a preferred embodiment, the binding moiety is a bispecific binding moiety comprising the binding site of a TCR and the binding site of an antibody. In a preferred embodiment the binding moiety is a bispecific T cell engaging receptor (TCER®) as disclosed in WO2019/012138A1.

In a preferred embodiment, the binding moiety is a bispecific binding moiety comprising the binding site of an antibody and a further binding site of another antibody.

Replacement Matrix for Mutational Scan (i.e. Step b)

The present method (also referred to herein as XPRES-Scan) uses a mutational scan (i.e. replacing the amino acids of the known target peptide of a binding moiety), which allows to identify additional (i.e. alternative) target peptides having a different amino acid sequence. In order to reduce the number of amino acid exchanges in the mutational scan not all positions in the amino acid sequence of the target peptide are subject to an amino acid exchange. Therefore a replacement matrix is generated that indicates the positions in the target peptide that are exchanged in the mutational scan. The number of amino acid exchanges indicated in the replacement matrix is lower than the number of all possible amino acid exchanges. The number of all possible amino acid exchanges results from the number of amino acids in the target peptide and the number of amino acids used in the mutational scan.

In a preferred embodiment, the alternative amino acids considered in the generation of the replacement matrix are selected from: alanine, glycine and proteinogenic amino acids, preferably from proteinogenic amino acids. In a preferred embodiment the replacement matrix does not include cysteine as an alternative amino acid in the mutational scan.

Furthermore, in the method of the present invention the selection of amino acids defined in the replacement matrix is not identical in all selections. For example, the replacement matrix does not indicate that each position is to be exchanged by the same amino acids, e.g. an alanine, a glycine etc.

In a preferred embodiment, the replacement matrix does not exclude a specific amino acid position of the target peptide (such as for example not including the anchor positions into the replacement matrix).

In a preferred embodiment, the replacement matrix is generated by determining for each amino acid of the target peptide the frequency of occurrence for every alternative amino acid in a suitable ligandome database.

In a preferred embodiment, the position specific occurrence for each alternative amino acid has a frequency of occurrence in the ligandome database of at least 2%, at least 1.5%, at least 1% or at least 0.5%, preferably at least 1% or at least 0.5%, to be included in the replacement matrix.

In a preferred embodiment, the position specific occurrence for each alternative amino acid is determined on how often they appear in the ligandome database (e.g. at least once, at least twice, at least three times). In a preferred embodiment the alternative amino acid occurs on a position at least once, at least twice, at least three times, at least four times (preferably at least once or twice). This embodiment is particularly advantageously for use with smaller ligandome databases or in case of an extremely sensitive mutational scan.

In general, the threshold for the frequency should be set low enough to include all amino acids that are reasonably likely to occur at a position but high enough to exclude amino acids that are present at a specific position in only very few peptides in the ligandome database and may be due to false-positive detections, e.g., during the mass-spectrometry analysis, or due to incorrect assignment of peptides to HLAs. In a preferred embodiment, the threshold for the frequency should be set such that each included combination of position and amino acid is supported by more than two peptides in the ligandome database. For example, if the ligandome database contains less than 400 peptides, the threshold for the frequency should be higher than 0.5%, because otherwise amino acids would be included based on only two detections. In larger ligandome databases, the threshold can be set lower to make the frequency-guidance more sensitive. An indication for a good threshold is that known anchor positions allow only few amino acids, whereas non-anchor positions allow for most amino acids.

In a preferred embodiment, the position specific occurrence for each alternative amino acid has a frequency of occurrence in the ligandome database of at least 1% to be included in the replacement matrix.

In a preferred embodiment, the position specific occurrence for each alternative amino acid has a frequency of occurrence in the ligandome database of at least 0.5% to be included in the replacement matrix.

Any ligandome database is suitable for use in the present invention that is derived from direct measurements of MHC peptides (preferably mass spectrometry following immunoprecipitation). Preferably the ligandome database provides a comprehensive coverage of major organs and tissues. Preferably the ligandome database includes MHC peptides from at least the following organs/tissues: blood cells, blood vessel, brain, heart, liver, lung, spinal cord, adipose tissue, adrenal gland, bile duct, bone, bone marrow, cartilage, central nerve, esophagus and stomach, eye, gallbladder, head-and-neck, kidney, large intestine, lymph node, pancreas, parathyroid gland, peripheral nerve, peritoneum, pituitary, pleura, skeletal muscle, skin, small intestine, spleen, stomach, thyroid gland, trachea, ureter, urinary bladder, breast, ovary, placenta, prostate, testis, thymus and uterus; preferably blood cells, blood vessel, brain, heart, liver, lung, spinal cord, adipose tissue, adrenal gland, bile duct, bone, bone marrow, cartilage, central nerve, esophagus and stomach, eye, gallbladder, head-and-neck, kidney, large intestine, lymph node, pancreas, parathyroid gland, peripheral nerve, peritoneum, pituitary, pleura, skeletal muscle, skin, small intestine, spleen, stomach, thyroid gland, trachea, ureter and urinary bladder; more preferably blood cells, blood vessel, brain, heart, liver, lung and spinal cord.

Preferably samples are primary tissues derived from healthy donors in order to avoid experimental bias (for instance due to culturing). Ideally, each organ should be covered by at least 5, at least 10, at least 15 donors, at least 20 donors, at least 25 donors, at least 30 donors (preferably at least 15 donors) to reflect biological variation and provide reasonable numbers of peptide sequences. Typically the number of donors also depends on the risk associated with particular organs and tissue. Some organs are associated with a high risk, a medium risk or a low risk. In this context risk refers to the potential relevance an off target in these organs/tissue may have.

Preferably, each organ/tissue with a low risk is represented by at least 5 donors; each organ/tissue with a medium risk is represented by at least 10 donors, and each organ/tissue with a high risk is represented by at least 20 donors.

Organs/tissues associated with a high risk are: blood cells, blood vessel, brain, heart, liver, lung and spinal cord. Organs/tissues associated with a medium risk are: adipose tissue, adrenal gland, bile duct, bone, bone marrow, cartilage, central nerve, esophagus and stomach, eye, gallbladder, head-and-neck, kidney, large intestine, lymph node, pancreas, parathyroid gland, peripheral nerve, peritoneum, pituitary, pleura, skeletal muscle, skin, small intestine, spleen, stomach, thyroid gland, trachea, ureter, urinary bladder. Organs/tissues associated with a low risk are: breast, ovary, placenta, prostate, testis, thymus and uterus.

Typically if less organs/tissues than the above mentioned high risk, medium risk and/or low risk tissues are covered the more donors should be included to cover the biological variance.

A non-limiting example of a suitable ligandome database is XPRESIDENT® (see Zhang, et al.; Nat Commun 9, 3919 (2018)) with 616 samples from healthy donors across 35 different organs capturing biological variation by 17.6 donors per organ.

Ligandome databases derived from mono-allelic cell-lines are sufficient to define binding motifs but a single cell-line is not comprehensive enough to capture biological variance introduced by organ and donor differences.

A further non-limiting example is the HLA ligand atlas (https://hla-ligand-atlas.org) which reflects biological variance across healthy organs and without culturing artefacts but covers individual differences on average only by 6.8 donors (198 class I samples for 29 organs).

In a preferred embodiment, the ligandome database combines liquid chromatography-mass spectrometry (LC-MS) for identification and quantitation of HLA ligands with RNA sequencing (RNA-seq) of corresponding mRNA from the same sample.

In a preferred embodiment, the ligands in the ligandome database are assigned to a single type of HLA.

In a preferred embodiment, the ligands in the ligandome database are experimentally confirmed binders (i.e. not only predicted binders).

In a preferred embodiment, the ligandome database is based on mass spectrometry data.

In a preferred embodiment, the ligandome database is HLA-specific. In other words, the ligandome database is specific for the HLA that presents the target peptide. In some embodiments the ligandome database includes data on more than one HLA.

In a preferred embodiment, the ligandome database is a ligandome database of MHC presented peptides, preferably of MHC 1 presented peptides.

In a preferred embodiment, the ligandome database comprises at least 200, 300, 400, 500, 600, 700, 800, 900, 1000 or more peptides.

In a preferred embodiment, the ligandome database comprises only peptides validated by mass spectrometry.

In a preferred embodiment, the ligandome database:
is a ligandome database of MHC presented peptides, preferably of MHC 1 presented peptides;
comprises at least 200, 300, 400, 500, 600, 700, 800, 900, 1000 or more peptides; and
comprises only peptides validated by mass spectrometry.

In a preferred embodiment the replacement matrix provides a selection of alternative amino acids for at least 5, at least 6, at least 7, at least 8, at least 9, each amino acid(s) of the target peptide. For example a target peptide having 10 amino acids on at least 5, at least 6, at least 7, at least 8, at least 9 or 10 positions an amino acid exchange is included in the replacement matrix.

Peptide Variants of the Target Peptide (i.e. Step c)

According to the replacement matrix peptide variants of the target peptide are generated. Each of these peptide variants only has one alternative amino acid as compared to the known target peptide. The peptide variants can be generated with any commonly known method to generated peptides of a specific sequence. Commonly known methods include solid-phase peptide synthesis (SPPS) such as Boc-SPPS or Fmoc-SPPS.

For each peptide variant at least one binding parameter of the peptide variant to the binding moiety is determined. The at least one binding parameter is selected from: binding affinity of the binding moiety to the peptide variant; association rate of the binding moiety to the peptide variant; dissociation rate of the binding moiety to the peptide variant; release of cytokines, preferably interferon γ, from a host cell expressing the binding moiety in response to binding the peptide variant; surface activation markers on a host cell expressing the binding moiety in response to binding the peptide variant. In a preferred embodiment the at least one binding parameter is identical for all peptide variants.

In a preferred embodiment the at least one binding parameter is the binding affinity of the binding moiety to the peptide variant. Methods for measuring the binding affinity include biolayer interferometry. In a preferred embodiment, the association and dissociation kinetics are determined. In a preferred embodiment, the dissociation constant of the peptide variant and the binding moiety is determined as binding parameter.

In a preferred embodiment, the binding parameter is a T cell functional assay. In a preferred embodiment, the T cell functional assay is selected from:
determining the release of cytokines, preferably interferon γ, from a T cell in response to binding the peptide variant;
determining the presence of surface activation markers on a T cell in response to binding the peptide variant;
determining the proliferation of a T cell in response to binding the peptide variant;
determining the cytotoxicity of a T cell in response to binding the peptide variant.

In a preferred embodiment, the T cell functional assay is determining the release of cytokines, preferably interferon γ, from a T cell in response to binding the peptide variant.

In a preferred embodiment, the T cell functional assay is determining the presence of surface activation markers on a T cell in response to binding the peptide variant.

In a preferred embodiment, the T cell functional assay is determining the proliferation of a T cell in response to binding the peptide variant.

In a preferred embodiment, the T cell functional assay is determining the cytotoxicity of a T cell in response to binding the peptide variant.

In a preferred embodiment the same at least one binding parameter determined for the peptide variants is also determined for the target peptide itself. In a preferred embodiment step c) or step I) further comprises determining at least one binding parameter of the target peptide to the binding moiety, and based thereon assigning a binding value to each amino acid of the target peptide, optionally wherein the position-specific binding value for each alternative amino acid of step d) i) or step I), is expressed in relation to the binding value of the amino acid of the target peptide.

In a preferred embodiment 2, 3, 4 or 5, preferably 2 or 3, binding parameters are determined for the peptide variants.

In a preferred embodiment, two binding parameters are determined for the peptide variants.

Alternative Aspect of Providing a Position-Specific Scoring Matrix (PSSM) (i.e. Alternative for Steps b) and c)

In an alternative aspect of the present invention instead of generating a replacement matrix (i.e. step b)) and perform a reduced mutational scan based thereon (i.e. step (c)), amino-acid similarity measures are used to construct a position-specific scoring matrix (PSSM) for identifying alternative target peptides. Optionally this can be combined with a mutational scan (preferably a reduced single amino acid replacement mutational scan).

In the context of the present invention any distance measure is also considered a similarity measure. Amino acid similarity measures are advantageous in the sense that they do not require wet-lab experiments (e.g. generating peptide variants and determining at least one binding parameter). Examples for amino-acid similarity measures that can be used include but are not limited to chemical properties, binding affinity data (e.g. PMBEC; see Kim et al; BMV Bioinformatics 2009; 10:394) and evolutionary relations between amino acids (e.g. BLOSUM; see Henikoff & Henikoff; PNAS 1992 Nov. 15; 89(22): 10915-10919). These similarity measures are used to construct a PSSM that provides a measure for each alternative amino acid similar to the at least one binding parameter of step c) in the first aspect of the invention.

In this alternative aspect each position in the PSSM corresponds to the similarity between the amino acid in the target peptide and the amino acid in the peptide variant. The resulting matrix (i.e. PSSM) is evaluated with the same scoring and ranking method (i.e. the second aspect of the present invention) that is used to evaluate the reduced mutational scan resulting from the replacement matrix of the first aspect of the present invention.

In a preferred embodiment the alternative aspect replaces steps b) and c) of the first aspect of the invention with the following steps:
b) generation of a position-specific scoring matrix (PSSM) for the target peptide, wherein the PSSM provides a selection of amino acids for each position of the target peptide;
c) assigning a value based on an amino acid similarity measure to each cell of the PSSM.

In other words this alternative aspect provides similar to the first aspect of the invention a replacement matrix (here position specific scoring matrix (PSSM)) including each position of the target peptide. In a preferred embodiment each position is replaced by any proteinogenic amino acid, i.e. the selection of amino acids includes any proteinogenic amino acid. This matrix is then filled with values (that correspond to the binding parameter of the first aspect of the invention) based on an amino acid similarity measure. For example if the replacement matrix indicates that each position is replaced by any other proteinongenic amino acid this would for a 9-mer target peptide result in a replacement matrix of 9×20 cells. Each of these cells is assigned a value from the amino acid similarity measure reflecting the change of the respective amino acids.

In a preferred embodiment the PSSM replaces each position of the target peptide with each proteinogenic amino acid and assigns a value to each cell that corresponds to the amino acid similarity measures (preferably PMBEC or BLOSUM) indicated for the specific amino acid replacement.

In a preferred embodiment the alternative aspect of the first aspect of the invention comprises the following steps:
a) providing a target peptide of the binding moiety;
b) generation of a position-specific scoring matrix (PSSM) for the target peptide, wherein the PSSM provides a selection of amino acids for each position of the target peptide;
c) assigning a value based on the amino acid similarity measure to each cell of the PSSM;
d) providing a list of potential alternative target peptides of the binding moiety comprising the following steps:
   i) calculating a position-specific binding value for the alternative amino acid present in the peptide variant of step c), based on the at least one binding parameter of the peptide variant calculated in step c),
   ii) providing a list of amino acid sequences having a specified length and being comprised in a protein database, preferably a proteome or ligandome database,
   iii) assigning an alternative target peptide value to each amino acid sequence of step ii) based on the position-specific binding values for each alternative amino acid present in the amino acid sequence;
   iv) selecting the potential alternative target peptides from the list of amino acid sequences of step ii) based on the binding peptide values assigned in step iii);
optional e) experimentally determining at least one binding parameter of the potential alternative target peptides to the binding moiety, wherein alternative target peptides are identified based on at least one binding parameter.

In a preferred embodiment the position-specific scoring matrix (PSSM) includes each position of the target peptide.

In a preferred embodiment the selection of alternative amino acids for each position of the target peptide in the position-specific scoring matrix (PSSM) includes each proteinogenic amino acids not being at the respective position in the target peptide.

In a preferred embodiment the PSSM does not include the anchor positions of the target peptide.

In a preferred embodiment the amino acid similarity measure is selected from chemical properties, binding affinity data (preferably peptide-MHC binding energy covariance (PMBEC)) and evolutionary relations between amino acids (preferably BLOSUM).

In a preferred embodiment the amino acid similarity measure is binding affinity data (preferably PMBEC).

In a preferred embodiment the amino acid similarity measure is evolutionary relations between amino acids (preferably BLOSUM).

In a preferred embodiment the amino acid similarity measure (preferably PMBEC or BLOSUM62) can also be combined with a mutational scan (preferably a single amino acid replacement mutational scan, e.g. an Ala-scan).

Identifying Potential Alternative Target Peptides (i.e. Step d)

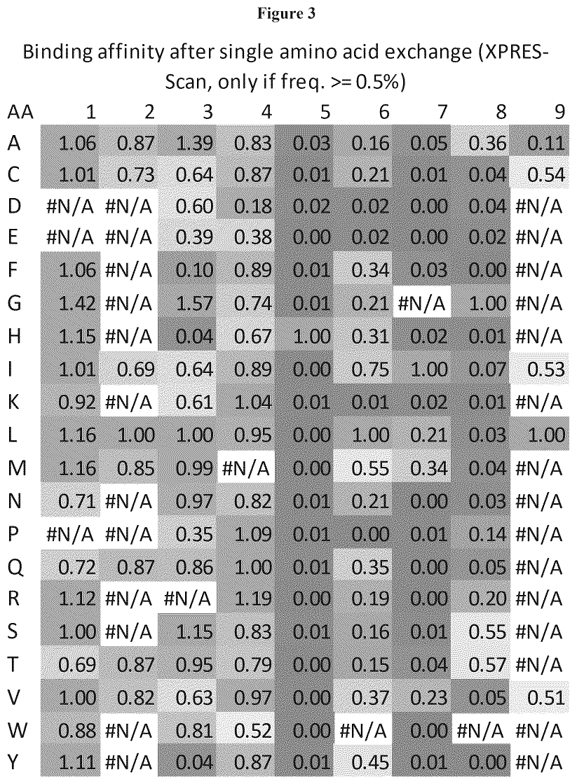

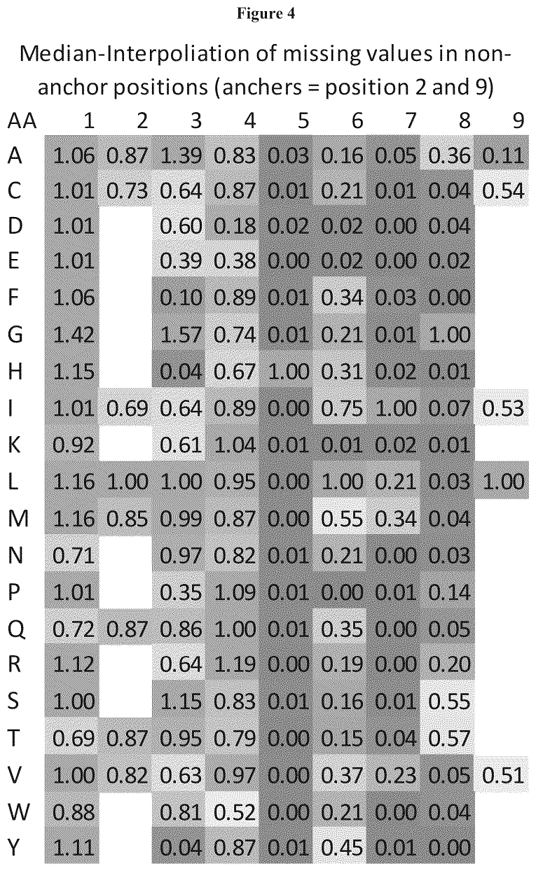

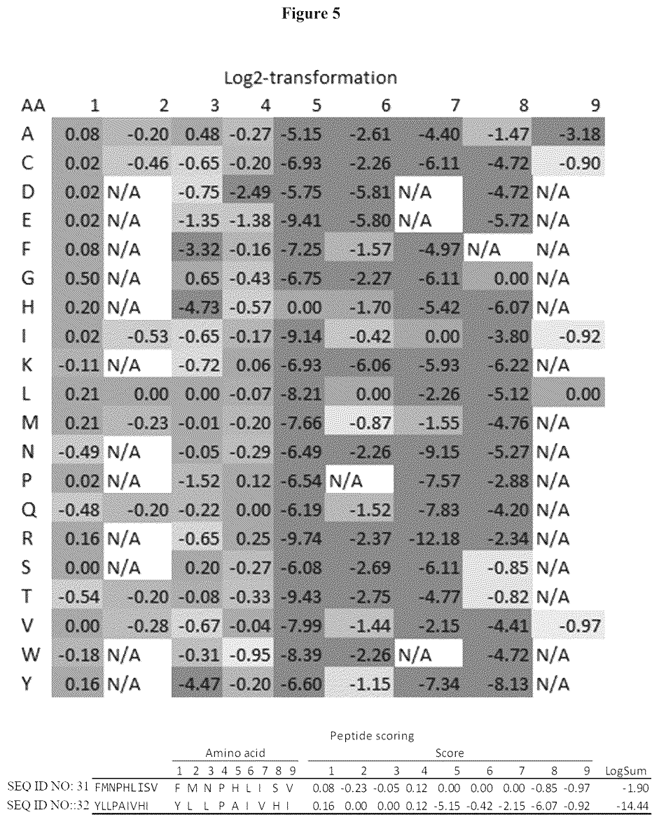

Based on the at least one binding parameter determined for the peptide variants a ranked list of potential alternative target peptides is generated. In a first step a position-specific binding value for the alternative amino acid present in the peptide variant is determined based on the at least one binding parameter of the peptide variant having an alternative amino acid at this position. For example, for a target peptide indicating in the replacement matrix 17 alternative amino acids at position 1, 17 peptide variants differing in the first amino acid are generated. After determining at least one binding parameter for each of the peptide variants the position specific binding values for position 1 would include 17 different values, i.e. one for each of the alternative amino acids present in the peptide variants (see also FIG. 3 having 17 different position specific binding values in position 1).

In a preferred embodiment, the position-specific binding value is determined in relation to the binding value measured for the target peptide. In other words, a relative binding value of 1 would indicate an identical position-specific binding value in the peptide variant than determined in the target peptide itself, whereas values below 1 indicate a decreased binding parameter and values above 1 indicate an increased binding parameter relative to the unmodified target peptide.

In a preferred embodiment each position-specific binding value of each possible alternative amino acid included in the selection of alternative amino acids of step b) that is below the limit of detection (LOD) of the method used to determine the at least one binding parameter in step c) is set to the LOD value of the respective method used (i.e. if the position-specific binding value for the alternative amino acid present in the peptide variant of step c) is lower than the LOD of the method used to determine the at least one binding parameter in step c), the position-specific binding value for the alternative amino acid is replaced by the LOD). A preferred method to determine the LOD of a method for determining at least one binding parameter of the peptide variant is to calculate the median of the standard deviations within each group of replicates used to measure the position-specific binding value for each alternative amino acid.

In a preferred embodiment, any position-specific binding values not determined for a particular amino acid that was not included in the replacement matrix are interpolated. For example a mutational scan performed for all 20 proteinogenic amino acids that only included 17 alternative amino acids in the replacement matrix at position one, would not have a position specific binding value assigned for 3 amino acids in position 1 (see also FIG. 3 showing binding values for 17 alternative amino acids at position 1). These missing position specific binding values can be interpolated from the measured binding values at this position.

In a preferred embodiment, the method used for interpolation is the median of all binding values measured at this position. The above example had 17 binding values determined at position 1 and did not provide a binding value for the remaining 3 positions. An interpolation using a median would calculate the median value of the 17 binding values at position 1 and assign this value to the seven missing amino acids in position 1.

In a preferred embodiment, the method used for interpolation is the use of the minimal value of all binding values measured at this position. In other words all missing values at a given position are filled with the minimal value measured at this position.

In a preferred embodiment the selection of alternative amino acids of step b) that is below the limit of detection (LOD) of the method used to determine the at least one binding parameter in step c) is set to the LOD value of the respective method used and the interpolation of missing values also takes the LOD values into consideration.

In a preferred embodiment, the interpolation, preferably using the median value or the minimum value or the LOD value, more preferably the LOD-value, is only applied in non-anchor positions of the peptide variant.

In a second step a list of amino acid sequences having a specified length and being comprised in a protein database, preferably a proteome or ligandome database, are provided. In this step a list of peptides should be provided that a) could theoretically be presented by the MHC/HLA presenting the target peptide and b) are known to be present in a particular organism.

In a preferred embodiment, the protein database represents the proteome of a particular organism. A preferred example of a suitable protein database representing the proteome is the UniProt protein database (www.uniprot.org). In some embodiments, only specific datasets comprised within the protein database (such as UniProt) are used in the method of the invention.

In a preferred embodiment, the datasets “Swiss Prot” and/or “Isoform sequences” of UniProt are used.

In a preferred embodiment, the protein database represents the ligandome of a particular MHC/HLA allotype, preferably the same MHC/HLA allotype the target peptide is bound to.

One preferred example of a suitable ligandome database is the HLA Ligand Atlas (hla-ligand-atlas.org).

In a preferred embodiment the specified length of the amino acid sequences is identical to that of the target peptide.

In a preferred embodiment the specified length of the amino acid sequences is 8, 9, 10, 11 or 12 amino acids.

In a preferred embodiment the specified length of the amino acid sequences is 8 amino acids.

In a preferred embodiment the specified length of the amino acid sequences is 9 amino acids.

In a preferred embodiment the specified length of the amino acid sequences is 10 amino acids.

In a preferred embodiment the specified length of the amino acid sequences is 11 amino acids.

In a preferred embodiment the specified length of the amino acid sequences is 12 amino acids.

In a preferred embodiment the specified length of the amino acid sequences depends on the length of the target peptide and the specified length is 1, 2, 3 or 4 amino acids shorter or longer than the target peptide.

In a preferred embodiment the specified length of the amino acid sequences is one amino acid longer than the length of the target peptide.

In a preferred embodiment the specified length of the amino acid sequences is one amino acid shorter than the length of the target peptide.

In a preferred embodiment the specified length of the amino acid sequences is two amino acids longer than the length of the target peptide.

In a preferred embodiment the specified length of the amino acid sequences is two amino acids shorter than the length of the target peptide.

In a preferred embodiment the specified length of the amino acid sequences is three amino acids longer than the length of the target peptide.

In a preferred embodiment the specified length of the amino acid sequences is three amino acids shorter than the length of the target peptide.

In a preferred embodiment the specified length of the amino acid sequences is four amino acids longer than the length of the target peptide.

In a preferred embodiment the specified length of the amino acid sequences is four amino acids shorter than the length of the target peptide.

The entries of the protein database are in silico digested to yield peptides having the specified length. In a preferred embodiment the in silico digest includes overlapping peptide sequences. In another embodiment, duplicates of the in silico digested peptides are removed from the list of amino acid sequences having a specified length.

In a preferred embodiment, the list of amino acid sequences having the specified length is further filtered by predicting the binding of the amino acid sequences to MHC/HLA, preferably MHC class I. Methods known to predict such binding are for example the NetMHC prediction algorithm.

In a third step an alternative target peptide value is assigned to each of the amino acid sequences determined in the second step (i.e. step d) ii)). This is based on the position specific binding values for alternative amino acids determined in the first step (i.e. step d) i)), which allows to determine an alternative target peptide value. For example, if in the second step the amino acid sequence IIVGAIGVGK was identified, then the position specific binding values for alternative amino acids determined in the first step would be combined, i.e. the values for isoleucine (I) at positions 1 and 2, valine (V) at position 3 etc. resulting in an alternative target peptide value for each amino acid sequence determined in the second step.

In a preferred embodiment the position-specific binding value for the alternative amino acid of the first step (i.e. d) i)) is logarithmized, preferably on a log 2 base, before assigning an alternative target peptide value to each amino acid sequence of step ii). In a preferred embodiment the position-specific binding value for the alternative amino acid of the first step (i.e. d) i)) are added according to the amino acids presented in the amino acid sequence determined in the second step.

In a preferred embodiment, the alternative target peptide value of step d) iii) is determined by calculating a sum of the logarithmic binding values of each amino acid present in the amino acid sequence of step ii).

In a preferred embodiment, the alternative target peptide value is calculated according to formula (I):

$${S}_{p} = {\sum }_{i = 1}^{9} {log}_{2} {B}_{{p}_{i} , i}$$

where S_{ρ} is the alternative target peptide value, ρᵢ is the amino acid at position i in the amino acid sequence of the second step ρ and B_{ρ}_{ᵢ}_{,i} is the position specific binding value determined by the mutational scan for the variant of the target peptide that has amino acid ρᵢ at position i.

In a preferred embodiment, the alternative target peptide value of step d) iii) is additionally based on:
a binding value for each amino acid of the target peptide that is present in the amino acid sequence; and
a position-specific binding value of each possible alternative amino acid not being included in the selection of alternative amino acids of step b) or the mutational scan of the target peptide, wherein the position-specific binding value is determined by the position-specific binding value of the alternative amino acids at the same position, preferably selected from a median, a mean, a minimum, and a maximum, more preferably a median or mean, most preferably a median.

In a preferred embodiment, the alternative target peptide value of step d) iii) is additionally based on a binding value for each amino acid of the target peptide that is present in the amino acid sequence.

In a preferred embodiment, the alternative target peptide value of step d) iii) is additionally based on a position-specific binding value of each possible alternative amino acid not being included in the selection of alternative amino acids of step b) or the mutational scan of the target peptide, wherein the position-specific binding value is determined by the position-specific binding value of the alternative amino acids at the same position, preferably selected from a median, a mean, a minimum, and a maximum, more preferably a median or mean, most preferably a median.

In a fourth step a potential alternative target peptide is selected from the list of amino acid sequences of the second step based on the binding peptide values assigned in the third step as described above.

In a preferred embodiment the potential alternative target peptides are selected by ranking the list of amino acid sequences of the second step by the alternative target peptide value assigned in the third step.

In a preferred embodiment, the potential alternative target peptides are selected by ranking the list of amino acid sequences of the second step by the alternative target peptide value assigned in the third step and then a number (e.g. 10, 20, 30, 40, 50, 60, 70, 80, 90, 100, 150, 200) of potential alternative target peptides are selected based on their ranking, with the highest ranking alternative target peptides being selected first. In a preferred embodiment the 10, 20, 30, 40, 50, 60, 70, 80, 90, 100, 150, or 200, preferably 10, 50 or 100, highest ranking potential alternative target peptides are selected. In an alternative preferred embodiment a variable number of potential alternative target peptides is selected up to a maximum until a pre-defined number of alternative targets is identified in step e) (e.g., testing up to 10, 20, 30, 40, 50, 60, 70, 80, 90, 100, 150, 200 potential alternative target peptides but stop further testing if one or more alternative target peptides have been identified. Typically the number of tested peptides may vary depending on many constraints, e.g. depending on the required “safety level” or the capacity for performing such tests. In a preferred embodiment of the alternative aspect of the invention using amino acid similarity measures the above indicated method for selecting a number of potential alternative target peptides from the ranked list is used.

In a preferred embodiment, the potential alternative target peptide is selected by ranking the list of amino acid sequences of the second step by the alternative target peptide values assigned in the third step. Then a cut-off value is applied and all amino acid sequences with an alternative target peptide value above the cut-off are considered as potential alternative target peptides. The cut-off is selected based on the need for sensitivity and specificity of the method. For example, a cut off of 50% loss of binding at a single position (but the loss may be distributed across all positions) is a very stringent cut-off. This cut off will result in few predicted off-targets that are very similar to the target peptide but may be very insensitive and could potentially miss relevant off-targets. In contrast, a cut-off of 50% loss of binding in seven positions is a low stringency cut-off that will result in more predicted off-targets and is thus potentially less specific. However, it will be more sensitive and could detect more relevant off-targets. The skilled person would determine the relevant cut off based on the particular needs of the assay being aware of the above consideration, in particular the balance between sensitivity and specificity.

In a preferred embodiment, the alternative target peptide value S_{ρ} is calculated according to formula (I) and the cut off C_{S} applied is in the range of −1 to −7, −2 to −6, −3 to −5 and −4 to −5, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −1, −2, −3, −4, −5, −6, −7, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −1, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −2, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −3, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −4, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −5, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −6, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −7, wherein a potential alternative target peptide is selected from the list of amino acid sequences of the second step if S_{ρ}≥C_{S}.

In case more than one binding parameter is determined, a position-specific binding value, alternative target peptide value and rank is determined separately for each binding parameter and the resulting ranks are combined afterwards for the selection of an alternative target binding peptide according to the methods disclosed herein.

Alternative Target Peptides (i.e. Step e))

In this step of the method for each potential alternative target peptide identified in the previous step at least one binding parameter to the binding moiety is determined. Alternative target peptides are identified based on at least one binding parameter. In general, the at least one binding parameter in this step is determined as described for step c) above.

The at least one binding parameter of the potential alternative target peptide to the binding moiety is selected from: binding affinity; association rate; dissociation rate; release of cytokines, preferably interferon γ, from a host cell expressing the binding moiety in response to binding the potential alternative target peptide; surface activation markers on a host cell expressing the binding moiety in response to binding the potential alternative target peptide. In a preferred embodiment, the at least one binding parameter is identical for all potential alternative target peptides

In a preferred embodiment, the same at least one binding parameter as in step c) is determined.

In a preferred embodiment the at least one binding parameter is the binding affinity of the binding moiety to the potential alternative target peptide. Methods for measuring the binding affinity include biolayer interferometry. In a preferred embodiment the association and dissociation kinetics are determined. In a preferred embodiment, the dissociation constant of the potential alternative target peptide and the binding moiety is determined as binding parameter.

In a preferred embodiment, the binding parameter is a T cell functional assay. In a preferred embodiment, the T cell functional assay is selected from:
determining the release of cytokines, preferably interferon γ, from a T cell in response to binding the potential alternative target peptide;
determining the presence of surface activation markers on a T cell in response to binding the potential alternative target peptide;
determining the proliferation of a T cell in response to binding the potential alternative target peptide;
determining the cytotoxicity of a T cell in response to binding the potential alternative target peptide.

In a preferred embodiment, the T cell functional assay is determining the release of cytokines, preferably interferon γ, from a T cell in response to binding the potential alternative target peptide.

In a preferred embodiment, the T cell functional assay is determining the presence of surface activation markers on a T cell in response to binding the potential alternative target peptide.

In a preferred embodiment, the T cell functional assay is determining the proliferation of a T cell in response to binding the potential alternative target peptide.

In a preferred embodiment, the T cell functional assay is determining the cytotoxicity of a T cell in response to binding the potential alternative target peptide.

In a preferred embodiment, 2, 3, 4 or 5, preferably 2 or 3, binding parameters are determined for the potential alternative target peptides.

In a preferred embodiment, 2 binding parameters are determined for the potential alternative target peptides.

In a preferred embodiment, the alternative target peptide is identified in step e) if the at least one binding parameter of step e) has a value of at least 50% of the same binding parameter determined for the target peptide.

In a preferred embodiment of the first aspect of the invention step e) is optional. In another preferred embodiment step e) is absent from the method of the first aspect of the invention.

### Second Aspect of the Invention

In a second aspect the present invention provides a method for the identification of alternative target peptides of a binding moiety based on a substitution analysis of a target peptide of said binding moiety, comprising the following steps:
I) determining a position-specific binding value for each alternative amino acid in the mutational scan of the target peptide, based on at least one binding parameter determined in the substitution analysis;
II) providing a list of amino acid sequences having a specified length and being comprised in a protein database, preferably a proteome or ligandome database;
III) assigning an alternative target peptide value to each amino acid sequence of step II) based on the position-specific binding values of each alternative amino acid of step I) present in the amino acid sequence;
IV) selecting the potential alternative target peptides from the list of amino acid sequences of step II) based on the alternative target peptide values assigned in step III);
V) determining at least one binding parameter of the potential alternative target peptides to the binding moiety, wherein alternative target peptides are identified based on at least one binding parameter.

The method of the second aspect represents the method used steps d) and e) in the first aspect of the invention. This method of the second aspect can however be used independently of the mutational scan using a replacement matrix or position-specific scoring matrix (PSSM) as disclosed in the first aspect of the invention. Different substitution analysis, such as for example a full mutational scan exchanging amino acids on every position of the target peptide, can be combined with the method of the second aspect of the invention.

In a first step a position-specific binding value for each alternative amino acid in the substitution analysis of the target peptide is determined based on at least one binding parameter determined in the substitution analysis (i.e. at least one binding parameter of the peptide variants to the binding moiety).

In a preferred embodiment, the substitution analysis is a full mutational scan. In such a full mutational scan for each amino acid position of the target peptide variants are generated in which the amino acid is replaced by every proteinogenic amino acid. In case of a target peptide with 9 amino acids this would result in 171 peptide variants, each differing in one amino acid from the target peptide. A full mutational scan further includes to determine for each of the peptide variants at least one binding parameter. The at least one binding parameter is determined as disclosed for the first aspect of the invention.

In a preferred embodiment, the position-specific binding value is determined in relation to the binding value measured for the target peptide. In other words, a relative binding value of 1 would indicate an identical position-specific binding value in the peptide variant than determined in the target peptide itself, whereas values below 1 indicate a decreased binding parameter and values above 1 indicate an increased binding parameter relative to the unmodified target peptide.

In a preferred embodiment each position-specific binding value of each possible alternative amino acid included in the mutational scan that is below the limit of detection (LOD) of the method used to determine the at least one binding parameter in the mutational scan is set to the LOD value of the respective method used (i.e. if the position-specific binding value for the alternative amino acid present in the mutational scan is lower than the LOD of the method used to determine the at least one binding parameter in the mutational scan, the position-specific binding value for the alternative amino acid is replaced by the LOD) A preferred method to determine the LOD of a method for determining at least one binding parameter of the peptide variant is to calculate the median of the standard errors within each group of replicates used to measure the position-specific binding value for each alternative amino acid.

In a preferred embodiment, not being based on a full mutational scan any position-specific binding values not determined for a particular amino acid are interpolated. For example a mutational scan performed for all 20 proteinogenic amino acids that only included 13 alternative amino acids in the mutational scan at position one, would not have a position specific binding value assigned for 7 amino acids in position 1. These missing position specific binding values can be interpolated from the measured binding values at this position.

In a preferred embodiment, the method used for interpolation is the median of all binding values measured at this position. The above example had 13 binding values determined at position 1 and did not provide a binding value for the remaining 7 positions. An interpolation using a median would calculate the median value of the 13 binding values at position 1 and assign this value to the seven missing amino acids in position 1.

In a preferred embodiment, the method used for interpolation is the use of the minimal value of all binding values measured at this position. In other words, all missing values at a given position are filled with the minimal value measured at this position.

In a preferred embodiment the selection of alternative amino acids of step b) that is below the limit of detection (LOD) of the method used to determine the at least one binding parameter in step c) is set to the LOD value of the respective method used and the interpolation of missing values also takes the LOD values into consideration.

In a preferred embodiment, the interpolation, preferably using the median value, the min value or the LOD-value, more preferably the LOD value, is only applied in non-anchor positions of the peptide variant.

In a second step a list of amino acid sequences having a specified length length and being compromised in a protein database, preferably a proteome or ligandome database, are provided. In this step a list of peptides should be provided that a) could theoretically be presented by the MHC/HLA presenting the target peptide and b) are known to be present in a particular organism.

In a preferred embodiment, the protein database represents the proteome of a particular organism. A preferred example of a suitable protein database representing the proteome is the UniProt protein database (www.uniprot.org). In some embodiments, only specific datasets comprised within the protein database (such as UniProt) are used in the method of the invention.

In a preferred embodiment, the datasets “Swiss Prot” and/or “Isoform sequences” of UniProt are used.

In a preferred embodiment, the protein database represents the ligandome of a particular MHC/HLA allotype, preferably the same MHC/HLA allotype the target peptide is bound to. One preferred example of a suitable ligandome database is the HLA Ligand Atlas (hla-ligand-atlas.org).

In a preferred embodiment the specified length of the amino acid sequences is identical to that of the target peptide.

In a preferred embodiment the specified length of the amino acid sequences is 8, 9, 10, 11 or 12 amino acids.

In a preferred embodiment the specified length of the amino acid sequences is 8 amino acids.

In a preferred embodiment the specified length of the amino acid sequences is 9 amino acids.

In a preferred embodiment the specified length of the amino acid sequences is 10 amino acids.

In a preferred embodiment the specified length of the amino acid sequences is 11 amino acids.

In a preferred embodiment the specified length of the amino acid sequences is 12 amino acids.

In a preferred embodiment the specified length of the amino acid sequences depends on the length of the target peptide and the specified length is 1, 2 or 3 amino acids short or longer than the target peptide.

In a preferred embodiment the specified length of the amino acid sequences is one amino acid longer than the length of the target peptide.

In a preferred embodiment the specified length of the amino acid sequences is one amino acid shorter than the length of the target peptide.

In a preferred embodiment the specified length of the amino acid sequences is two amino acids longer than the length of the target peptide.

In a preferred embodiment the specified length of the amino acid sequences is two amino acids shorter than the length of the target peptide.

In a preferred embodiment the specified length of the amino acid sequences is three amino acids longer than the length of the target peptide.

In a preferred embodiment the specified length of the amino acid sequences is three amino acids shorter than the length of the target peptide.

The entries of the protein database are in silico digested to yield peptides having the specified length. In a preferred embodiment the in silico digest includes overlapping peptide sequences. In another embodiment, duplicates of the in silico digested peptides are removed from the list of amino acid sequences having a specified length.

In a preferred embodiment, the list of amino acid sequences having the specified length is further filtered by predicting the binding of the amino acid sequences to MHC/HLA, preferably MHC class I. Methods known to predict such binding are for example the NetMHC prediction algorithm.

In a third step an alternative target peptide value is assigned to each of the amino acid sequences determined in the second step (i.e. step II)). This is based on the position specific binding values for alternative amino acids determined in the first step (i.e. step I)), which allows to determine an alternative target peptide value. For example, if in the second step the amino acid sequence IIVGAIGVGK (SEQ ID NO: 25) was identified, then the position specific binding values for alternative amino acids determined in the first step would be combined, i.e. the values for isoleucine (I) at positions 1 and 2, valine (V) at position 3 etc. resulting in an alternative target peptide value for each amino acid sequence determined in the second step.

In a preferred embodiment the position-specific binding value for the alternative amino acid of the first step (i.e. I)) is logarithmized, preferably on a log 2 base, before assigning an alternative target peptide value to each amino acid sequence of step II). In a preferred embodiment the position-specific binding value for the alternative amino acid of the first step (i.e. I)) are added according to the amino acids presented in the amino acid sequence determined in the second step.

In a preferred embodiment, the alternative target peptide value of step III) is determined by calculating a sum of the logarithmic binding values of each amino acid present in the amino acid sequence of step II).

In a preferred embodiment, the alternative target peptide value is calculated according to formula (I):

$${S}_{p} = {\sum }_{i = 1}^{9} {log}_{2} {B}_{{p}_{i} , i}$$

where S_{ρ} is the alternative target peptide value, ρᵢ is the amino acid at position i in the amino acid sequence of the second step ρ and B_{ρ}_{ᵢ}_{,i} is the position specific binding value determined by the mutational scan for the variant of the target peptide that has amino acid ρᵢ at position i.

In a preferred embodiment, the alternative target peptide value of step III) is additionally based on:
a binding value for each amino acid of the target peptide that is present in the amino acid sequence; and
a position-specific binding value of each possible alternative amino acid not being included in the selection of alternative amino acids of the mutational scan of the target peptide, wherein the position-specific binding value is determined by the position-specific binding value of the alternative amino acids at the same position, preferably selected from a median, a mean, a minimum, and a maximum, more preferably a median or mean, most preferably a median.

In a preferred embodiment, the alternative target peptide value of step III) is additionally based on a binding value for each amino acid of the target peptide that is present in the amino acid sequence.

In a preferred embodiment, the alternative target peptide value of step III) is additionally based on a position-specific binding value of each possible alternative amino acid not being included in the selection of alternative amino acids of the mutational scan of the target peptide, wherein the position-specific binding value is determined by the position-specific binding value of the alternative amino acids at the same position, preferably selected from a median, a mean, a minimum, and a maximum, more preferably a median or mean, most preferably a median.

In a fourth step a potential alternative target peptide is selected from the list of amino acid sequences of the second step based on the binding peptide values assigned in the third step as described above.

In a preferred embodiment the potential alternative target peptides are selected by ranking the list of amino acid sequences of the second step by the alternative target peptide value assigned in the third step.

In a preferred embodiment, the potential alternative target peptides are selected by ranking the list of amino acid sequences of the second step by the alternative target peptide value assigned in the third step and then a number (e.g. 10, 20, 30, 40, 50, 60, 70, 80, 90, 100, 150, 200) of potential alternative target peptides are selected based on their ranking, with the highest ranking alternative target peptides being selected first. In a preferred embodiment the 10, 20, 30, 40, 50, 60, 70, 80, 90, 100, 150, or 200, preferably 10, 50 or 100, highest ranking potential alternative target peptides are selected. In an alternative preferred embodiment a variable number of potential alternative target peptides is selected up to a maximum until a pre-defined number of alternative targets is identified in step e) (e.g., testing up to 10, 20, 30, 40, 50, 60, 70, 80, 90, 100, 150, 200 potential alternative target peptides but stop further testing if one or more alternative target peptides have been identified. Typically the number of tested peptides may vary depending on many constraints, e.g. depending on the required “safety level” or the capacity for performing such tests. In a preferred embodiment of the alternative aspect of the invention using amino acid similarity measures the above indicated method for selecting a number of potential alternative target peptides from the ranked list is used.

In a preferred embodiment, the potential alternative target peptide is selected by ranking the list of amino acid sequences of the second step by the alternative target peptide values assigned in the third step. Then a cut-off value is applied and all amino acid sequences with a alternative target peptide value above the cut-off are considered as potential alternative target peptides. The cut-off is selected based on the need for sensitivity and specificity of the method. For example, a cut off of 50% loss of binding at a single position (but the loss may be distributed across all positions) is a very stringent cut-off. This cut off will result in few predicted off-targets that are very similar to the target peptide but may be very insensitive and could potentially miss relevant off-targets. In contrast, a cut-off of 50% loss of binding in seven positions is a low stringency cut-off that will result in more predicted off-targets and is thus potentially less specific. However, it will be more sensitive and could detect more relevant off-targets. The skilled person would determine the relevant cut off based on the particular needs of the assay being aware of the above consideration, in particular the balance between sensitivity and specificity.

In a preferred embodiment, the alternative target peptide value S_{ρ} is calculated according to formula (I) and the cut off C_{S} applied is in the range of −1 to −7, −2 to −6, −3 to −5 and −4 to −5, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −1, −2, −3, −4, −5, −6, −7, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −1, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −2, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −3, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −4, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −5, wherein a potential alternative target peptide is selected from the list of amino acid sequences of the second step if S_{ρ}≥C_{S}.

In a preferred embodiment, the alternative target peptide value S_{ρ} is calculated according to formula (I) and the cut off C_{S} applied is −6, wherein a potential alternative target peptide is selected from the list of amino acid sequences of the second step if S_{ρ} C_{S}.

In a preferred embodiment, the alternative target peptide value S_{ρ} is calculated according to formula (I) and the cut off C_{S} applied is −7, wherein a potential alternative target peptide is selected from the list of amino acid sequences of the second step if S_{ρ} C_{S}.

In case more than one binding parameter is determined, a position-specific binding value, alternative target peptide value and rank is determined separately for each binding parameter and the resulting ranks are combined afterwards for the selection of an alternative target binding peptide according to the methods disclosed herein.

In a fifth step for each potential alternative target peptide identified in the previous step at least one binding parameter to the binding moiety is determined. Alternative target peptides are identified based on at least one binding parameter. In general, the at least one binding parameter in this step is determined as described for the mutational scan above.

The at least one binding parameter of the potential alternative target peptide to the binding moiety is selected from: binding affinity; association rate; dissociation rate; release of cytokines, preferably interferon γ, from a host cell expressing the binding moiety in response to binding the potential alternative target peptide; surface activation markers on a host cell expressing the binding moiety in response to binding the potential alternative target peptide. In a preferred embodiment, the at least one binding parameter is identical for all potential alternative target peptides

In a preferred embodiment, the same at least one binding parameter as in the mutational scan is determined.

In a preferred embodiment the at least one binding parameter is the binding affinity of the binding moiety to the potential alternative target peptide. Methods for measuring the binding affinity include biolayer interferometry. In a preferred embodiment the association and dissociation kinetics are determined. In a preferred embodiment, the dissociation constant of the potential alternative target peptide and the binding moiety is determined as binding parameter.

In a preferred embodiment, the binding parameter is a T cell functional assay. In a preferred embodiment, the T cell functional assay is selected from:
determining the release of cytokines, preferably interferon γ, from a T cell in response to binding the potential alternative target peptide;
determining the presence of surface activation markers on a T cell in response to binding the potential alternative target peptide;
determining the proliferation of a T cell in response to binding the potential alternative target peptide;
determining the cytotoxicity of a T cell in response to binding the potential alternative target peptide.

In a preferred embodiment, the T cell functional assay is determining the release of cytokines, preferably interferon γ, from a T cell in response to binding the potential alternative target peptide.

In a preferred embodiment, the T cell functional assay is determining the presence of surface activation markers on a T cell in response to binding the potential alternative target peptide.

In a preferred embodiment, the T cell functional assay is determining the proliferation of a T cell in response to binding the potential alternative target peptide.

In a preferred embodiment, the T cell functional assay is determining the cytotoxicity of a T cell in response to binding the potential alternative target peptide.

In a preferred embodiment, 2, 3, 4 or 5, preferably 2 or 3, binding parameters are determined for the potential alternative target peptides.

In a preferred embodiment, 2 binding parameters are determined for the potential alternative target peptides.

In a preferred embodiment, the alternative target peptide is identified in step e) if the at least one binding parameter of step e) has a value of at least 50% of the same binding parameter determined for the target peptide.

In a preferred embodiment of the second aspect of the invention step V) is optional. In another preferred embodiment step V) is absent from the method of the second aspect of the invention.

### Examples

### Example 1: Mutational Scan of Target Peptide for Bispecific Binding Moiety (TCER #1)

### 1.1 Production and Purification of Soluble Bispecific Molecules (TCER #1)

The present invention was exemplified using a bispecific antigen binding proteins directed against a major histocompatibility (MHC) presented target peptide. The bispecific antigen binding protein comprises two antigen binding sites, wherein the first antigen binding site binds to a target peptide/MHC complex and the second binding site binds to CD3. The first binding site is a TCR derived binding site, whereas the second binding site originates from an antibody. The production of the bispecific binding moiety used in this example (TCER #1) is explained in the following paragraph.

Vectors for the expression of recombinant proteins were designed as monocistronic pUC19-derivatives controlled by HCMV-derived promoter elements. Plasmid DNA was amplified in E. coli according to standard culture methods and subsequently purified using commercial-available kits (Macherey & Nagel). Purified plasmid DNA was used for transient transfection of CHO-S cells according to instructions of the manufacturer (ExpiCHO™ system; Thermo Fisher Scientific). Transfected CHO-cells were cultured for 6-14 days at 32° C. to 37° C. and received one to two feeds of ExpiCHO™ Feed solution. Conditioned cell supernatant was cleared by filtration (0.22 m) utilizing Sartoclear Dynamics@Lab Filter Aid (Sartorius). Bispecific molecule TCER #1 (polypeptide 1 with amino acid sequence of SEQ ID NO: 1, polypeptide 2 with amino acid sequence of SEQ ID NO: 13) was purified using an Akta Pure 25 L FPLC system (GE Lifesciences) equipped to perform affinity and size-exclusion chromatography in line. Affinity chromatography was performed on protein A or L columns (GE Lifesciences) following standard affinity chromatographic protocols. Size exclusion chromatography was performed directly after elution (pH 2.8) from the affinity column to obtain highly pure monomeric protein using Superdex 200 pg 16/600 columns (GE Lifesciences) following standard protocols. Protein concentrations were determined on a NanoDrop system (Thermo Scientific) using calculated extinction coefficients according to predicted protein sequences. Concentration was adjusted, if needed, by using Vivaspin devices (Sartorius). Finally, purified molecules were stored in phosphate-buffered saline at concentrations of about 1 mg/mL at temperatures of 2-8° C. Quality of purified bispecific molecules was determined by HPLC-SEC on MabPac SEC-1 columns (5 pm, 7.8×300 mm) running in 50 mM sodium-phosphate pH 6.8 containing 300 mM NaCl within a Vanquish UHPLC-System. Non-reducing and reducing SDS-PAGE confirmed the purity and expected size of TCER #1.

TABLE 1
Amino acid sequence of TCER #1

| SEQ | | |
|---|---|---|
| ID | | |
| NO: | Function | Sequence |
| 1 | polypeptide 1 | QKEVEQNSGPLSVPEGAIASLNCTYSDRGSQSFFWYRQYSGKSPELIMSIYQEGD |
| | | KEDGRFTAQLNKASQYVSLLIRDSQPSDSATYLCAAVIDNDQGGILTFGTGTRLT |
| | | IIPNIQNGGGSGGGGDIQMTQSPSSLSASVGDRVTITCRASQDIRNYLNWYQQKP |
| | | GKAPKLLIYYTSRLHSGVPSRFSGSGSGTDYTLTISSLQPEDIATYFCQQGQTLP |
| | | WTFGQGTKVEIKEPKSSDKTHTCPPCPAPPVAGPSVFLFPPKPKDTLMISRTPEV |
| | | TCVVVDVSHEDPEVKFNWYVDGVEVHNAKTKPREEQYQSTYRVVSVLTVLHQDWL |
| | | NGKEYKCKVSNKALPASIEKTISKAKGQPREPQVYTLPPCRDELTKNQVSLWCLV |
| | | KGFYPSDIAVEWESNGQPENNYKTTPPVLDSDGSFFLYSKLTVDKSRWQQGNVFS |
| | | CSVMHEALHNHYTQKSLSLSP |
| 2 | polypeptide | QKEVEQNSGPLSVPEGAIASLNCTYSDRGSQSFFWYRQYSGKSPELIMSIYQEGD |
| | 1_alpha chain | KEDGRFTAQLNKASQYVSLLIRDSQPSDSATYLCAAVIDNDQGGILTFGTGTRLT |
| | | IIPNIQN |
| 3 | polypeptide | DRGSQS |
| | 1_CDRal | |
| 4 | polypeptide | IYQEGD |
| | 1_CDRa2 | |
| 5 | polypeptide | CAAVIDNDQGGILTF |
| | 1_CDRa3 | |
| 6 | polypeptide | QKEVEQNSGPLSVPEGAIASLNCTYS |
| | 1_FRa1 | |
| 7 | polypeptide | FFWYRQYSGKSPELIMS |
| | 1_FRa2 | |
| 8 | polypeptide | KEDGRFTAQLNKASQYVSLLIRDSQPSDSATYL |
| | 1_FRa3 | |
| 9 | polypeptide | GTGIRLTIIPNIQN |
| | 1_FRa4 | |
| 10 | polypeptide | GGGSGGGG |
| | 1_linker | |
| 11 | polypeptide | DIQMTQSPSSLSASVGDRVTITCRASQDIRNYLNWYQQKPGKAPKLLIYYTSRLH |
| | 1_antiCD3 VL | SGVPSRFSGSGSGTDYTLTISSLQPEDIATYFCQQGQTLPWTFGQGTKVEIK |
| 12 | polypeptide | EPKSSDKTHTCPPCPAPPVAGPSVFLFPPKPKDTLMISRTPEVTCVVVDVSHEDP |
| | 1_Fc region | EVKFNWYVDGVEVHNAKTKPREEQYQSTYRVVSVLTVLHQDWLNGKEYKCKVSNK |
| | | ALPASIEKTISKAKGQPREPQVYTLPPCRDELTKNQVSLWCLVKGFYPSDIAVEW |
| | | ESNGQPENNYKTTPPVLDSDGSFFLYSKLTVDKSRWQQGNVFSCSVMHEALHNHY |
| | | TQKSLSLSP |
| 13 | polypeptide 2 | EVQLVQSGAEVKKPGASVKVSCKASGYSFTGYTMNWVRQAPGQGLEWMGLINPYK |
| | | GVSTYAQKFQDRVTLTVDKSTSTAYMELSSLRSEDTAVYYCARSGYYGDSDWYFD |
| | | VWGQGTLVTVSSGGGSGGGGKAGVTQTPRYLIKTRGQQVTLSCSPIPGHRAVSWY |
| | | QQTPGQGLQFLFEYVHGEERNKGNFPGRFSGRQFSNSSSEMNISNLELGDSALYL |
| | | CASSPWDSPNVQYFGPGTRLTVTEDLKNEPKSSDKTHTCPPCPAPPVAGPSVELF |
| | | PPKPKDTLMISRTPEVTCVVVDVSHEDPEVKENWYVDGVEVHNAKTKPREEQYQS |
| | | TYRVVSVLTVLHQDWLNGKEYKCKVSNKALPASIEKTISKAKGQPREPQVCTLPP |
| | | SRDELTKNQVSLSCAVKGFYPSDIAVEWESNGQPENNYKTTPPVLDSDGSFFLVS |
| | | KLTVDKSRWQQGNVFSCSVMHEALHNHYTQKSLSLSP |
| 14 | polypeptide | KAGVTQTPRYLIKTRGQQVTLSCSPIPGHRAVSWYQQTPGQGLQFLFEYVHGEER |
| | 2_beta chain | NKGNFPGRESGRQFSNSSSEMNISNLELGDSALYLCASSPWDSPNVQYFGPGTRL |
| | | TVTEDLKN |
| 15 | polypeptide | PGHRA |
| | 2_CDRb1 | |
| 16 | polypeptide | YVHGEE |
| | 2_CDRb2 | |
| 17 | polypeptide | CASSPWDSPNVQYF |
| | 2_CDRb3 | |
| 18 | polypeptide | KAGVTQTPRYLIKTRGQQVTLSCSPI |
| | 2_FRb1 | |
| 19 | polypeptide | VSWYQQTPGQGLQFLFE |
| | 2_FRb2 | |
| 20 | polypeptide | RNKGNFPGRFSGRQFSNSSSEMNISNLELGDSALYL |
| | 2_FRb3 | |
| 21 | polypeptide | GPGTRLTVTEDLKN |
| | 2_FRb4 | |
| 22 | polypeptide | GGGSGGGG |
| | 2_linker | |
| 23 | polypeptide | EVQLVQSGAEVKKPGASVKVSCKASGYSFTGYTMNWVRQAPGQGLEWMGLINPYK |
| | 2_antiCD3 | GVSTYAQKFQDRVTLTVDKSTSTAYMELSSLRSEDTAVYYCARSGYYGDSDWYFD |
| | VH | VWGQGTLVTVSS |
| 24 | polypeptide 2_ | EPKSSDKTHTCPPCPAPPVAGPSVFLFPPKPKDTLMISRTPEVTCVVVDVSHEDP |
| | Fc region | EVKFNWYVDGVEVHNAKTKPREEQYQSTYRVVSVLTVLHQDWLNGKEYKCKVSNK |
| | | ALPASIEKTISKAKGQPREPQVCTLPPSRDELTKNQVSLSCAVKGFYPSDIAVEW |
| | | ESNGQPENNYKTTPPVLDSDGSFFLYSKLTVDKSRWQQGNVFSCSVMHEALHNHY |
| | | TQKSLSLSP |

### 1.2 Mutational Scan

For each position (1 to 9) of the peptide sequence, 18 proteinogenic amino acids (all but cysteine) different from the target amino acid were substituted resulting in 162 peptide variants. Cysteine was not substituted because the synthesis of peptides containing cysteine was not possible due to chemical properties of the thiol side chain. Peptides were synthetized in house in 0.5 μmol scale with a filter tip-based approach on a Syro II synthesizer (Multisyntech, Witten, Germany) using solid phase standard Fmoc-chemistry.

### 1.3 Binding Data

To test for all possible amino acid exchanges the effect on binding, TCER #1 was analyzed for his binding affinity towards HLA-A*02/PRAME-004 and HLA-A*02/PRAME-004 variants via biolayer interferometry. Measurements were performed on an Octet RED384 system using settings recommended by the manufacturer. Briefly, binding kinetics were measured at 30° C. and 1000 rpm shake speed using PBS, 0.05% Tween-20, 0.1% BSA as buffer. Disulfide-stabilized HLA-A*02 molecules with histidine tag were incubated with PRAME-004 peptide or PRAME-004 peptide variants prior to loading of the complexes onto HIS1K biosensors at a concentration of 10 μg/ml. As reference sensor, HLA-A*02 complexes with the unrelated peptide DDX5-001 were loaded onto the biosensors. Serial dilutions of the TCER #1 (15 nM, 50 nM, 158 nM and 500 nM) were incubated with the bio sensors to determine association and dissociation kinetics. The dissociation constant for HLA-A*02/PRAME-004 was determined and set to 1 (reflecting a 100% binding reference). Using the dissociation constants for all PRAME-004 variants a relative dissociation compared to HLA-A*02/PRAME-004 was calculated and used for the generation of a binding matrix in which values of 1 reflect the identical dissociation constant than for HLA-A*02/PRAME-004 while values below and above 1 reflect a higher and a lower dissociation constant. Since per variant 1 mutation occurred the corresponding value was then assigned accordingly in the binding matrix. Each variant was measured in triplicate.

### 1.4 Experimental Confirmation of Binding Peptides

To confirm experimentally off-targets for the TCER #1 a binding capture assay was performed as disclosed in WO 2021/028503A1. Briefly TCER #1 was coupled to a solid Sepharose® matrix at a pre-determined ratio using chemical coupling after BrCN activation. In parallel the same amount of Sepharose® was also activated for coupling using the same strategy, yet without addition of TCER #1. Instead a 0.1 M solution of the amino acid glycine was added to the Sepharose®, which instead coupled to the chemically activated groups. T98G cells were subjected to lysis in a CHAPS detergent containing buffer and homogenized assisted by sonification. T98G cells expressing the potential alternative target peptides were subjected to lysis in a CHAPS detergent containing buffer and homogenized assisted by sonification. The T98G lysate containing the mixture of the potential alternative target peptides was then applied to two affinity chromatography columns loaded with 200 μI of the glycine coupled Sepharose® matrix or 200 μI of the Sepharose® matrix coupled with TCER #1. The T98G derived lysate was thereby applied in such a fashion that it would first be run over the glycine coupled Sepharose® (referred to herein as glycine column) to remove or deplete any peptides, which would bind non-specifically to the column or the Sepharose® matrix before the isolation of peptides which bind to TCER #1. After washing of the affinity columns with PBS and ddH2O the bound potential alternative target peptides were eluted from the columns using Trifluoroacetic acid (TFA).

During this step, MHC bound peptides are also released from the MHC moiety and can be separated from higher molecular weight molecules by ultrafiltration using specified devices with a molecular weight cutoff of less than 10 kDa.

The isolated peptide mixtures were then finally subjected to liquid chromatography coupled mass spectrometry (LC-MS) using a nanoACQUITY UPLC system (Waters) followed by an Orbitrap fusion™ Tribrid™ mass spectrometer (Thermo Scientific).

Mass spectrometry instruments were operated in data-dependent mode (ODA) utilizing different fragmentation techniques (in this example, CID and HCD fragmentation) as well as MS/MS spectra readout in two different analyzers (in this example, Ion Trap and Orbitrap analyzers).

Peptide fragment spectra were searched against the human proteome using a modified version of the International protein index (IPI v.378) and the Universal protein resource (UniProt) sequence database with the search engine SEQUEST. All peptides eluted and identified from the glycine column were excluded as these represent non-specific binding peptides. Furthermore, known contaminants according to in-house databases and algorithms for their identification were removed from the analysis.

All identified peptides were subjected to binding kinetics measurements with bio-layer interferometry using an Octet RED384 system, Briefly, binding kinetics were measured at 30° C. and 1000 rpm shake speed using PBS, 0.05% Tween-20, 0.1% BSA as buffer. Peptide:MHC complexes were loaded onto biosensors (HIS1K) prior to analyzing serial dilutions of the TCER #1.

All peptides having a KD<100 in reference to the target PRAME were identified as binding peptides. Those binding peptides were used as a ground truth for the bioinformatic analysis in example 2.

### Example 2: Comparison of Predictive Model Building Using Data of Example 1

### 2.1 Predictive Model (XPRES-Scan)

### 2.1.1 HLA-Specific Position-Wise Amino Acid Frequency

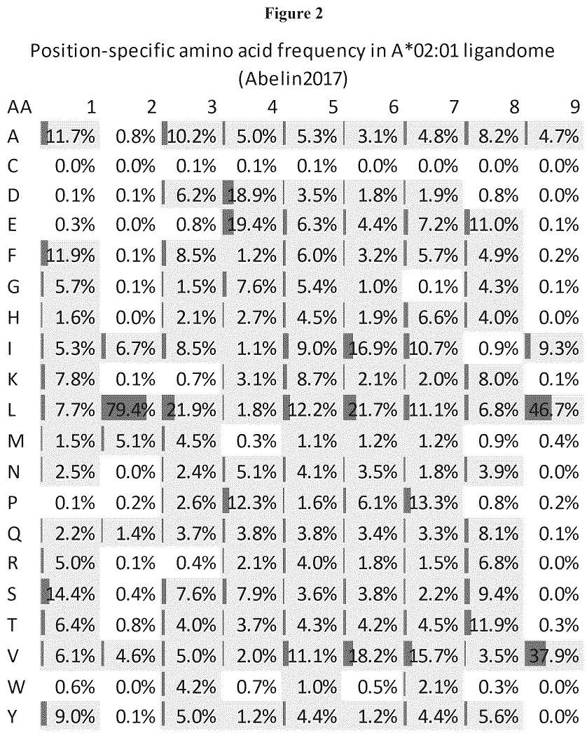

To determine the position-specific amino acid frequency in peptides presented by the target HLA, we used a previously published HLA-specific reference ligandome that was determined with mass-spectrometry of mono-allelic cell-lines (Abelin et al 2017, Immunity 46, 315-326). The reference ligandome was filtered to peptides of the same length as the target peptide (9 amino acids) and the frequency of each of the 20 proteinogenic amino acids was determined at each position by counting the occurrences of the amino acid at a position and dividing by the total number of peptides of length 9 in the reference ligandome (see FIG. 2).

### 2.1.2 Determination of Anchor Position

The target HLA of the evaluated TCER is HLA A*02:01, for which the anchor positions 2 and the C-terminus (i.e., position 9 for peptides of length 9) have commonly been reported in the literature. This is supported by the position-wise amino acid frequency determined in 2.1.1. At the known anchor positions 2 and 9, only 7 (position 2) and 4 (position 9) amino acids have a frequency above a frequency cut-off of 0.5% (5 and 4 amino acids at position 2 and 9, respectively, for a frequency cut-off of 1%).

### 2.1.3 Mutational Scan

A frequency-guided mutational scan excludes mutated variants of the target peptide that are mutated with an amino acid at a position that has a position-specific frequency below a chosen cut-off, e.g., 0.5% or 1%.

The threshold should be set low enough to include all amino acids that are reasonably likely to occur at a position but high enough to exclude spurious amino acids, i.e., amino acids that are present at a specific position in only very few peptides in the database and may be due to false-positive detections, e.g., during the mass-spectrometry analysis, or due to incorrect assignment of peptides to HLAs. In general, the threshold should be set such that each included combination of position and amino acid is supported by more than two peptides to avoid spurious detections. For example, if the reference database (i.e. ligandome database) contains less than 400 peptides, the threshold should be higher than 0.5%, because otherwise amino acids would be included based on only two detections. In larger databases, the threshold can be set lower to make the frequency-guidance more sensitive. An indication for a good threshold is that known anchor positions (e.g., from literature) allow only few amino acids, whereas non-anchor positions allow for most amino acids. Based on these considerations, we found 0.5% and 1% to yield reasonable matrices for frequency guidance.

To simulate a frequency-guided mutational scan, we removed from the full mutational scan performed in Example 1 those mutated variants that would not be included in a frequency-guided mutational scan. For example, the amino acid aspartic acid (symbol Asp or D) has a frequency of 0.06% at position 1 in the reference ligandome. Therefore, it would not be measured in a frequency-guided mutational scan and the value measured in the full mutational scan was set to “not available” (N/A) in the simulated frequency-guided mutational scan (see FIG. 3 as an example).

Because the full mutational scan performed in Example 1 did not include cysteine substitutions, the values for cysteine were interpolated with the median of the binding value of all substituted amino acids.

### 2.1.4 Interpolation of Missing Values in Mutation Scan

To interpolate the binding of the mutational variants that were not measured by the frequency-guided mutational scan, the N/A values are imputed with the median of the binding value of the measured mutational variants for each non-anchor position (1,3,4,5,6,7,8) individually. For example, the binding value for aspartic acid at position 1, which was not measured by the frequency-guided mutational scan (see 2.1.3), was imputed as the median of all measured mutational variants that were mutated at position 1 (including the non-mutated target peptide).

To demonstrate the effects of different imputation methods, we alternatively imputed missing values with the minimum of the binding value of the measured mutational variants or with a LOD of about 5%. This LOD value was determined based on the median of the standard deviations of the binding values for each mutational variant measured in example 1, section 1.3. Specifically, each binding value is measured in triplicate and expressed in relation to the binding value of the original target peptide (i.e. as a percentage). The mean of the three measured values is the final binding value of the variant. For each of these triplicates (one per variant), the sample standard deviation was calculated. In example 1, section 1.3 171 variants were measured resulting in 171 sample standard deviations. The method error is then estimated as the median of these 171 sample standard deviation, which is about 5%. This 5% method error was then used as the LOD.

The mutational variants with amino-acid exchanges in anchor positions that were not measured by the frequency-guided mutational scan were not imputed.

The interpolation of N/A values in non-anchor positions results in an interpolated frequency-guided mutational scan, which was used to calculate the binding score for putative off-targets.

### 2.1.5 UniProt Protein Database Digestion

To determine the list of peptides that could theoretically be presented by the target HLA, we downloaded the UniProt protein database (datasets “Reviewed (Swiss-Prot)” and “Isoform sequences”, release version 2020_06¹) in FASTA format which contains 604,375 protein sequences. We merged the two datasets and filtered it to include only proteins of Homo sapiens (tax ID 9606) which resulted in a total of 42,383 human protein sequences. All proteins in the merged dataset were in-silico digested into 9-mers (amino acids of length 9). For the in-silico digest, we included overlapping 9-mers. For example, the protein sequence “MTMDKSELVQK” (SEQ ID NO: 26) was digested into the 9-mers “MTMDKSELV” (SEQ ID NO: 27), “TMDKSELVQ” (SEQ ID NO:,28) and “MDKSELVQK” (SEQ ID NO: 29). The resulting 9-mers were sorted and uniquified. This resulted in 10,797,420 unique 9-mers. ¹ https://www.uniprot.org/downloads #uniprotkblink, accessed on 2021-02-10

### 2.1.6 Binding Score Calculation

For each peptide p in the digested UniProt peptide database, we calculated the binding score S_{ρ} as

$${S}_{p} = {\sum }_{i = 1}^{9} {log}_{2} {B}_{{p}_{i} , i}$$

where ρᵢ is the amino acid at position i in peptide ρ and B_{ρ}_{ᵢ}_{,i} is the binding value determined by the mutational scan for the mutational variant that has amino acid ρᵢ at positioni.

### 2.1.7 Alternative Target Peptide Selection

To determine if a peptide ρ is a predicted off-target, the binding score S_{ρ} is compared with a cut-off. We evaluated different cut-offs C_{S} between −1 and −7 to select predicted off-targets, i.e., all peptide with binding score S_{ρ}≥C_{S} are predicted to be off-targets. A cut-off of −1 in log-space can be interpreted as a 50% loss of binding at a single position (but the loss may be distributed across all positions). This is a very stringent cut-off that will result in few predicted off-targets that are very similar to the target peptide but may be very insensitive and could potentially miss relevant off-targets. A cut-off of −7 can be interpreted as a 50% loss of binding in seven positions (e.g., all but the two anchor positions). This is a more lenient cut-off that will result in more predicted off-targets and is thus potentially less specific. However, it will be more sensitive and could detect more relevant off-targets.

To determine the number of true alternative target peptides that would be identified by measuring the binding value of a fixed number of potential alternative target peptides, we sorted the peptides in the UniProt database by their binding score calculated in 2.1.6 and determined the number of true alternative target peptides (see Example 1, 1.4).

### Results

The table below shows the results obtained with amino-acid-frequencies based on Abelin et al., 2017, mutational scan with frequency cut-off of 0.5%, interpolation of non-anchor positions with the median. The search for potential off-targets was done in the 9-mer-digested UniProt protein database (datasets “Reviewed (Swiss-Prot)” and “Isoform sequences”, release version 2020_06¹), in the UniProt database after pre-filterering with NetMHC 3.0 (predicted HLA A*02:01 affinity≤500 nM) following the procedure disclosed in Karapetyan et al., and in the XPRESIDENT® ligandome database (see Zhang, et al.; Nat Commun 9, 3919 (2018)). Across all threshold and methods, the best results are achieved with the XPRESIDENT® ligandome database, followed by the UniProt database filtered with NetMHC (see also table 18 for a comparison of PRC-AUCS). ¹ https://www.uniprot.org/downloads #uniprotkblink, accessed on 2021-02-10

TABLE 2

| | | #Potential | | | |
|---|---|---|---|---|---|
| | | alternative | | | |
| Interpolation | | target | #True | | |
| method | C_{S} | peptides | positives | Recall | Precision |
| Median | −1 | 6 | 1 | 0.07 | 0.17 |
| | −2 | 18 | 5 | 0.33 | 0.28 |
| | −3 | 57 | 8 | 0.53 | 0.14 |
| | −4 | 189 | 11 | 0.73 | 0.06 |
| | −5 | 445 | 11 | 0.73 | 0.02 |
| | −6 | 884 | 12 | 0.80 | 0.01 |
| | −7 | 1700 | 13 | 0.87 | 0.01 |
| LOD | −1 | 6 | 1 | 0.07 | 0.17 |
| | −2 | 16 | 5 | 0.33 | 0.31 |
| | −3 | 45 | 8 | 0.53 | 0.18 |
| | −4 | 156 | 11 | 0.73 | 0.07 |
| | −5 | 485 | 12 | 0.80 | 0.02 |
| | −6 | 1421 | 13 | 0.87 | 0.01 |
| | −7 | 3700 | 13 | 0.87 | 0.00 |
| minimum | −1 | 6 | 1 | 0.07 | 0.17 |
| | −2 | 16 | 5 | 0.33 | 0.31 |
| | −3 | 47 | 8 | 0.53 | 0.17 |
| | −4 | 157 | 11 | 0.73 | 0.07 |
| | −5 | 374 | 11 | 0.73 | 0.03 |
| | −6 | 746 | 11 | 0.73 | 0.01 |
| | −7 | 1388 | 12 | 0.80 | 0.01 |

The columns indicate:
C_{S}: The used cut-off for the binding score S_{ρ}.
# Potential alternative target peptides: Number of peptides in the search database that have a binding score above the cut-off.
# True positives: Number of predicted off-targets that are validated off-targets (see Example 1).
Recall: Proportion of all validated alternative target peptides (n=15) that was predicted by the method. This is the primary metric for comparing method performance.
Precision: Proportion of predicted alternative target peptides that are alternative target peptides. This is a measure of the specificity of the method and is the secondary metric for comparing method performance. Of note reporting the specificity is not meaningful here due to the large number of negative samples (i.e., non-off-targets, n=10,797,406) compared to the number of positive samples (off-targets, n=15)

Table 3 below shows the results obtained with amino-acid-frequencies based on Abelin et al., 2017, mutational scan with frequency cut-off of 0.5%, interpolation of non-anchor positions with the median. The search for potential off-targets was done in the 9-mer-digested UniProt protein database (datasets “Reviewed (Swiss-Prot)” and “Isoform sequences”, release version 2020_06¹). In contrast to the above disclosed example no cut-off value was applied but instead a fixed number of potential alternative target peptides were tested starting with the highest ranking potential alternative target peptide. ¹ https://www.uniprot.org/downloads #uniprotkblink, accessed on 2021-02-10

TABLE 3

| | #Potential | | | |
|---|---|---|---|---|
| Interpolation | alternative | | | |
| method | target peptides | #True positives | Recall | Precision |
| median | 10 | 2 | 0.13 | 0.20 |
| | 20 | 6 | 0.40 | 0.30 |
| | 50 | 7 | 0.47 | 0.14 |
| | 100 | 8 | 0.53 | 0.08 |
| | 200 | 11 | 0.73 | 0.06 |
| LOD | 10 | 2 | 0.13 | 0.20 |
| | 20 | 6 | 0.40 | 0.30 |
| | 50 | 8 | 0.53 | 0.16 |
| | 100 | 8 | 0.53 | 0.08 |
| | 200 | 11 | 0.73 | 0.06 |
| minimum | 10 | 2 | 0.13 | 0.20 |
| | 20 | 6 | 0.40 | 0.30 |
| | 50 | 8 | 0.53 | 0.16 |
| | 100 | 8 | 0.53 | 0.08 |
| | 200 | 11 | 0.73 | 0.06 |

The columns indicate:
# Potential alternative target peptides: The number of peptides (soiled by binding value) for which binding is measured starting with the highest ranking potential alternative target peptide.
# True positives: Number of potential alternative target peptides that are validated alternative target peptides (see Example 1).
Recall: Proportion of all validated alternative target peptides (n=15) that were predicted by the method. This is the primary metric for comparing method performance.
Precision: Proportion of identified peptides that are alternative target peptides. This is a measure of the specificity of the method and is the secondary metric for comparing method performance. Of note reporting the specificity is not meaningful here due to the large number of negative samples (i.e., non-off-targets, n=10,797,406) compared to the number of positive samples (off-targets, n=15)

Table 4 below shows the results obtained with amino-acid-frequencies based on Abelin et al., 2017, mutational scan with frequency cut-off of 0.5%, interpolation of non-anchor positions with the median. The search for potential off-targets was done in the 9-mer-digested UniProt protein database (datasets “Reviewed (Swiss-Prot)” and “Isoform sequences”, release version 2020_06¹). The 9-mer were pre-filtered with NetMHC 3.0 (predicted HLA A*02:01 affinity≤500 nM) following the procedure disclosed in Karapetyan et al. ¹ https://www.uniprot.org/downloads #uniprotkblink, accessed on 2021-02-10

TABLE 4

| | | #Predicted | | | |
|---|---|---|---|---|---|
| | | alternative | | | |
| Interpolation | | target | #True | | |
| method | C_{S} | peptides | positives | Recall | Precision |
| Median | −1 | 5 | 1 | 0.07 | 0.20 |
| | −2 | 10 | 5 | 0.33 | 0.50 |
| | −3 | 29 | 8 | 0.53 | 0.28 |
| | −4 | 86 | 11 | 0.73 | 0.13 |
| | −5 | 183 | 11 | 0.73 | 0.06 |
| | −6 | 326 | 12 | 0.80 | 0.04 |
| | −7 | 601 | 13 | 0.87 | 0.02 |
| LOD | −1 | 5 | 1 | 0.07 | 0.20 |
| | −2 | 9 | 5 | 0.33 | 0.56 |
| | −3 | 27 | 8 | 0.53 | 0.30 |
| | −4 | 85 | 11 | 0.73 | 0.13 |
| | −5 | 249 | 12 | 0.80 | 0.05 |
| | −6 | 716 | 12 | 0.80 | 0.02 |
| | −7 | 1844 | 12 | 0.80 | 0.01 |
| minimum | −1 | 5 | 1 | 0.07 | 0.20 |
| | −2 | 9 | 5 | 0.33 | 0.56 |
| | −3 | 27 | 8 | 0.53 | 0.30 |
| | −4 | 80 | 11 | 0.73 | 0.14 |
| | −5 | 170 | 11 | 0.73 | 0.06 |
| | −6 | 302 | 11 | 0.73 | 0.04 |
| | −7 | 536 | 12 | 0.80 | 0.02 |

Table 5 below differs from Table 4 above in that no cut-off value was applied but instead a fixed number of potential alternative target peptides were tested starting with the highest ranking potential alternative target peptide.

TABLE 5

| | #Potential | | | |
|---|---|---|---|---|
| Interpolation | alternative | | | |
| method | target peptides | #True positives | Recall | Precision |
| median | 10 | 5 | 0.33 | 0.50 |
| | 20 | 6 | 0.40 | 0.30 |
| | 50 | 8 | 0.53 | 0.16 |
| | 100 | 11 | 0.73 | 0.11 |
| | 200 | 11 | 0.73 | 0.06 |
| LOD | 10 | 6 | 0.40 | 0.60 |
| | 20 | 6 | 0.40 | 0.30 |
| | 50 | 8 | 0.53 | 0.16 |
| | 100 | 11 | 0.73 | 0.11 |
| | 200 | 12 | 0.80 | 0.06 |
| Minimum | 10 | 6 | 0.40 | 0.60 |
| | 20 | 6 | 0.40 | 0.30 |
| | 50 | 8 | 0.53 | 0.16 |
| | 100 | 11 | 0.73 | 0.11 |
| | 200 | 11 | 0.73 | 0.73 |

The table 6 below shows the results obtained with amino-acid-frequencies based on Abelin et al., 2017, mutational scan with frequency cut-off of 0.5%, interpolation of non-anchor positions with the median. The search for potential off-targets was done in the XPRESIDENT® ligandome database (see Zhang, et al.; Nat Commnun 9, 3919 (2018)).

TABLE 6

| | | #Predicted | | | |
|---|---|---|---|---|---|
| | | alternative | | | |
| Interpolation | | target | #True | | |
| method | C_{S} | peptides | positives | Recall | Precision |
| Median | −1 | 1 | 1 | 0.07 | 1 |
| | −2 | 6 | 5 | 0.33 | 0.83 |
| | −3 | 10 | 8 | 0.53 | 0.8 |
| | −4 | 26 | 11 | 0.73 | 0.42 |
| | −5 | 48 | 11 | 0.73 | 0.23 |
| | −6 | 66 | 12 | 0.8 | 0.18 |
| | −7 | 128 | 13 | 0.87 | 0.1 |
| LOD | −1 | 1 | 1 | 0.07 | 1.00 |
| | −2 | 5 | 5 | 0.33 | 1.00 |
| | −3 | 9 | 8 | 0.53 | 0.89 |
| | −4 | 26 | 11 | 0.73 | 0.42 |
| | −5 | 59 | 12 | 0.80 | 0.20 |
| | −6 | 158 | 13 | 0.87 | 0.08 |
| | −7 | 363 | 13 | 0.87 | 0.04 |
| minimum | −1 | 1 | 1 | 0.07 | 1.00 |
| | −2 | 5 | 5 | 0.33 | 1.00 |
| | −3 | 9 | 8 | 0.53 | 0.89 |
| | −4 | 24 | 11 | 0.73 | 0.46 |
| | −5 | 45 | 11 | 0.73 | 0.24 |
| | −6 | 64 | 11 | 0.73 | 0.17 |
| | −7 | 117 | 12 | 0.80 | 0.10 |

Table 7 below differs from Table 6 above in that no cut-off value was applied but instead a fixed number of potential alternative target peptides were tested starting with the highest ranking potential alternative target peptide.

TABLE 7

| | #Potential | | | |
|---|---|---|---|---|
| Interpolation | alternative | | | |
| method | target peptides | #True positives | Recall | Precision |
| median | 10 | 8 | 0.53 | 0.80 |
| | 20 | 10 | 0.67 | 0.50 |
| | 50 | 11 | 0.73 | 0.22 |
| | 100 | 13 | 0.87 | 0.13 |
| | 200 | 14 | 0.93 | 0.07 |
| LOD | 10 | 8 | 0.53 | 0.80 |
| | 20 | 10 | 0.67 | 0.50 |
| | 50 | 12 | 0.80 | 0.24 |
| | 100 | 13 | 0.87 | 0.13 |
| | 200 | 13 | 0.87 | 0.07 |
| minimum | 10 | 8 | 0.53 | 0.80 |
| | 20 | 10 | 0.67 | 0.50 |
| | 50 | 11 | 0.73 | 0.22 |
| | 100 | 12 | 0.80 | 0.12 |
| | 200 | 14 | 0.93 | 0.07 |

2.2 Comparison with Full-Scan as Described in the Art

The use of a full-scan to identify off-targets had been described in Maier et al. 2000 (Eur. J. Immunol., 30: 448-457), Harper et al. ((2018) PLoS ONE 13(10):e0205491) and WO2014/096803A1. The full-scan uses a mutational scan that replaces each position of the target peptide with every amino acid to estimate the loss of binding for each mutational variant of the target peptide (for a total of 171 mutational variants for a 9-mer peptide). The mutational scan is then filtered with a pre-defined cut-off to identify “tolerated” amino acids. The tolerated amino acids are used to generate a binding motif that can be used to perform a “motif search” in a peptide or sequence databases to identify potential off-targets. A motif search compares each peptide or sequence in a database with the binding motif. If each amino acids of the peptide is allowed by the motif (i.e., tolerated according to the mutational scan), the peptide or sequence is a “match” and a potential off-target. In WO2014/096803A1, the authors propose binding losses of 50%, 60%, up to 90% as potential thresholds. Thus, we evaluated the cut-offs of 0.5 (i.e., 50% loss of binding), 0.4 (i.e., 60% loss of binding), 0.3, 0.2 and 0.1.

In contrast, the method of the present invention (XPRES-Scan) does not perform a full mutational scan and uses a binding score to identify potential off-targets. Instead of the full mutation scan, XPRES-Scan only measures the loss of binding for amino acids that pass the HLA-specific position-wise frequency thresholds (see section 2.1.3 above). Also, XPRES-Scan does not perform a motif-search but assigns a binding score (that is calculated based on the mutational scan) to each peptide or sequence in the search database and uses a cut-off to identify potential off-targets.

### Results

The motif search was performed against the UniProt protein database (datasets “Reviewed (Swiss-Prot)” and “Isoform sequences”, release version 2020_06¹). ¹ https://www.uniprot.org/downloads #uniprotkblink, accessed on 2021-02-10

The PRC-AUC of the full-scan (PRC-AUC=0.074) is lower than the PRC-AUC of the XPRES-Scan on both the XPRESIDENT® ligandome (PRC-AUC=0.571) and the UniProt protein database (PRC-AUC=0.177) indicating a better performance of the XPRES-Scan.

The recall of the full-can is lower than the recall of the predictive model at the best cut-off for all evaluated cut-offs and the precision is also lower, i.e., the full-scan does not identify as many true off-targets as the predictive model and predicts many more false-positive off-targets that need to be followed up on with post-hoc tests.

TABLE 8

| | #Potential | | | |
|---|---|---|---|---|
| | alternative target | | | |
| Cut-off | peptides | #True positives | Recall | Precision |
| 0.5 | 102 | 2 | 0.13 | 0.02 |
| 0.4 | 125 | 2 | 0.13 | 0.02 |
| 0.3 | 556 | 7 | 0.47 | 0.01 |
| 0.2 | 2,953 | 9 | 0.60 | 0.00 |
| 0.1 | 10,167 | 11 | 0.73 | 0.00 |

2.3 Comparison with a Partial-Scan as Described in the Art

The use of a partial-scan to identify off-targets had been described in Karapetyan et al (Front. Immunol., 22 Oct. 2019). We performed the analysis here as described in the prior art except that we used the UniProt database instead of the NCBI Reference Sequences Database (RefSeq) to make the results more comparable with XPRES-Scan and full-scan. We evaluated the cut-offs 0.905 and 0.85 that are proposed in the publication and lower cut-offs than those proposed (i.e. 0.8, 0.75, 0.7 and 0.65) to evaluate if lower cut-offs improve the recall.

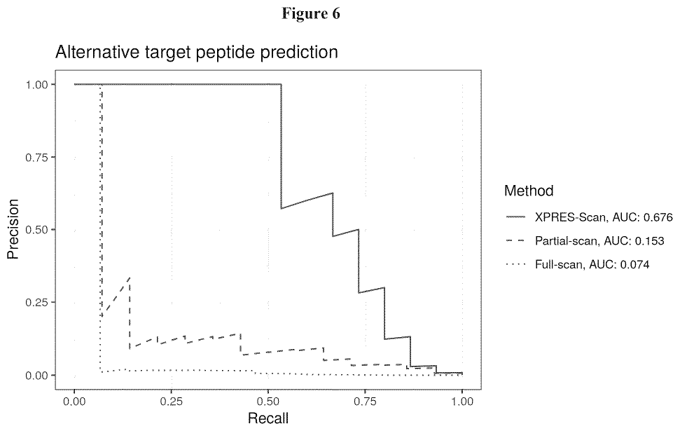

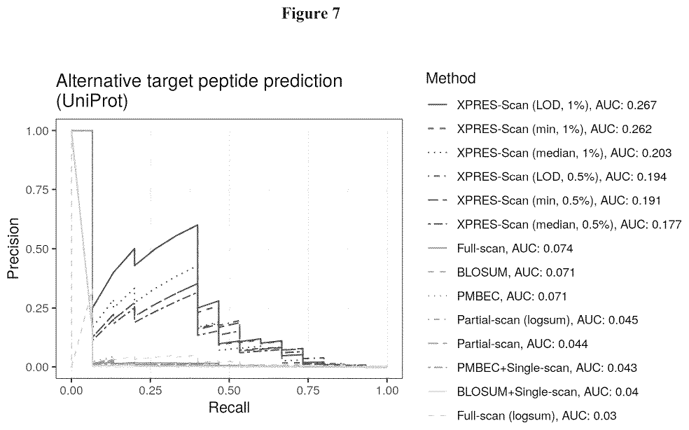

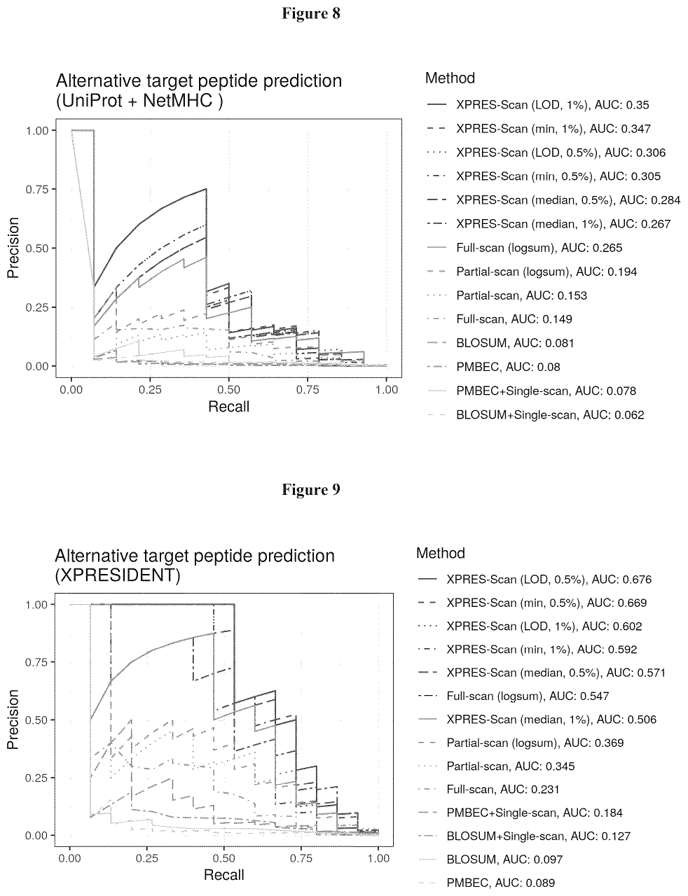

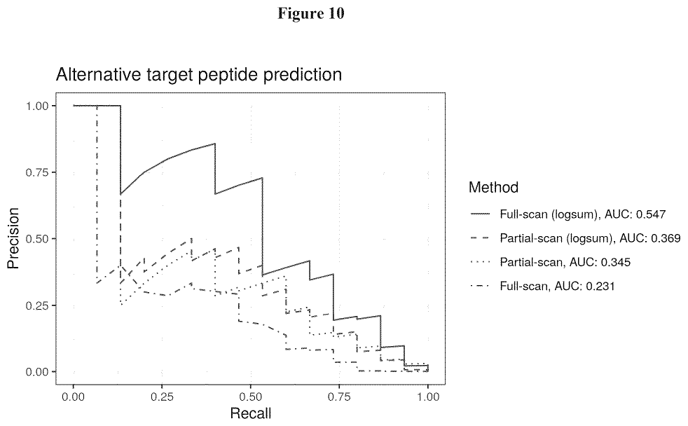

To compare the partial-scan globally, i.e. without applying a specific cut-off, we created a precision-recall curve (PRC) and calculated the area under the PRC (PRC-AUC) (FIG. 6).

### Result

The search was performed against the 9-mer-digested UniProt protein database (datasets “Reviewed (Swiss-Prot)” and “Isoform sequences”, release version 2020_06¹). The 9-mer were pre-filtered with NetIHC 3.0 (predicted HLA A*02:01 affinity≤500 nM) following the procedure disclosed in Karapetyan et al.

For both recommended cut-offs (i.e. 0.905 and 0.85), the recall is below the recall (i.e. sensitivity) of the XPRES-Scan at the best cut-off (0.87; see Table 2 above). At the recommended cut-off, the precision is higher for the partial-scan compared with the best cut-off of the presented predictive model but the recall is much lower. If we compare cut-offs of XPRES-Scan and the partial-scan that have a comparable recall, XPRES-Scan achieves consistently higher precision.

We also looked at cut-offs lower than those proposed by Karapetyan et al (i.e. 0.8, 0.75, 0.7 and 0.65) to check if lower cut-offs improve the recall. For cut-offs of 0.7 and below, the recall is the same or better than most sensitive cut-off of XPRES-Scan (87%) but the precision is much lower.

TABLE 9

| | #Predicted | | | |
|---|---|---|---|---|
| | alternative target | #True | | |
| Cut-off | peptides | positives | Recall | Precision |
| 0.905 | 9 | 2 | 0.13 | 0.22 |
| 0.850 | 87 | 6 | 0.40 | 0.07 |
| 0.800 | 278 | 10 | 0.67 | 0.04 |
| 0.750 | 1,019 | 13 | 0.87 | 0.01 |
| 0.700 | 4,466 | 14 | 0.93 | 0.00 |
| 0.650 | 11,712 | 14 | 0.93 | 0.00 |

### Example 3: Comparison of Predictive Model Building Using Data of Douglas et al. (2021)

To validate our results of Example 2 with a structurally different binding moiety (i.e. a bispecific antibody), we repeated the analysis described above with a second off-target search described by Douglas et al. (2021) in Science Immunology, Vol. 6, Issue 57. Douglas et al. performed a mutational scan for a bispecific antibody targeting mutant RAS-neoantigens. The neoantigen is an HLA A*03:01-specific 10-mer that originates from the G12V mutational variant of the KRAS gene from codon 7 to 16 (VVVGAVGVGK) (SEQ ID NO: 30).

### 3.1 Predictive Model

### 3.1.1 HLA-Specific Position-Wise Amino Acid Frequency

We determined the position-wise amino acid frequency as described in 2.1.1 with a target length of 10 amino acids. Because the ligandome database published by Abelin et al. used to determine the amino acid frequencies contains only 388 HLA-A*03:01-specific 10-mers, we used a cut-off of 1% (in contrast to Example 2). A cut-off of 0.5% would include amino acids that are supported by only two peptides and therefore would be prone to spurious peptides that may not reflect the actual HLA-specific amino acid binding motif.

### 3.1.2 Determination of Anchor Position

The target HLA of the evaluated bispecific antibody is HLA A*03:01, for which the anchor positions 2 and the C-terminus (i.e., position 10 for peptides of length 10) have commonly been reported in the literature.

### 3.1.3 Mutational Scan

The mutational scan was extracted from the supplementary material of Douglas et al. (2021).

### 3.1.4 Interpolation of Missing Values in Mutation Scan

Interpolation of missing values in mutation scan was performed as described in 2.1.4

### 3.1.5 UniProt Protein Database Digestion

UniProt protein database digestion was performed as described in 2.1.5 with a target length of 10 amino acids. This resulted in 10,867,795 unique 10-mers.

### 3.1.6 Binding Score Calculation and Off-Target Selection

Binding score calculation and off-target selection were performed as described in 2.1.6 and 2.1.7.

### Result

Table 10 below shows the results obtained with amino-acid-frequencies based on Abelin et al., 2017, mutational scan with frequency cut-off of 1%, interpolation of non-anchor positions with the median. The search for potential off-targets was done in the 10-mer-digested UniProt protein database (only human proteins, with isoforms). Because Douglas et al. reported only one true off-target for the validation, we only show the number of predicted off-targets and if the off-target was found or not. The optimal cut-off for this off-target search is −2, at which the off-target is detected and only 5 off-targets are predicted in total.

TABLE 10

| Interpolation | | #Predicted off− | Off-target |
|---|---|---|---|
| method | C_{S} | targets | found? |
| Median | −1 | 0 | No |
| | −2 | 5 | Yes |
| | −3 | 11 | Yes |
| | −4 | 31 | Yes |
| | −5 | 71 | Yes |
| | −6 | 159 | Yes |
| | −7 | 329 | Yes |
| LOD | −1 | 0 | No |
| | −2 | 4 | Yes |
| | −3 | 9 | Yes |
| | −4 | 28 | Yes |
| | −5 | 89 | Yes |
| | −6 | 229 | Yes |
| | −7 | 476 | Yes |
| Minimum | −1 | 0 | No |
| | −2 | 4 | Yes |
| | −3 | 9 | Yes |
| | −4 | 28 | Yes |
| | −5 | 63 | Yes |
| | −6 | 145 | Yes |
| | −7 | 289 | Yes |

Table 11 below shows the results obtained with amino-acid-frequencies based on Abelin et al., 2017, mutational scan with frequency cut-off of 1%, interpolation of non-anchor positions with the median. The search for potential off-targets was done in the 10-mer-digested UniProt protein database (only human proteins, with isoforms). The 9-mer were pre-filtered with NetMC 3.0 (predicted HLA A*02:01 affinity≤500 nMV) following the procedure disclosed in Karapetyan et al.

TABLE 11

| Interpolation | | #Predicted off− | Off-target |
|---|---|---|---|
| method | C_{S} | targets | found? |
| Median | −1 | 0 | No |
| | −2 | 5 | Yes |
| | −3 | 11 | Yes |
| | −4 | 29 | Yes |
| | −5 | 59 | Yes |
| | −6 | 118 | Yes |
| | −7 | 224 | Yes |
| LOD | −1 | 0 | No |
| | −2 | 4 | Yes |
| | −3 | 9 | Yes |
| | −4 | 26 | Yes |
| | −5 | 78 | Yes |
| | −6 | 193 | Yes |
| | −7 | 377 | Yes |
| Minimum | −1 | 0 | No |
| | −2 | 4 | Yes |
| | −3 | 9 | Yes |
| | −4 | 26 | Yes |
| | −5 | 56 | Yes |
| | −6 | 114 | Yes |
| | −7 | 209 | Yes |

The alternative target peptide was not found in the XPRESIDENT ligandome database because it was never detected with mass spectrometry. Given the broad coverage of human tissues in the XPRESIDENT ligandome database, this indicates that the alternative target peptide is very likely not presented on human tissue cells and therefore may not be relevant for the risk assessment.

3.2 Comparison with Full-Scan as Described in the Art

The use of a full-scan to identify cross targets had been described in Maier et al. 2000, Harper et al. (2018) and WO2014/096803A1 and section 2.2. We evaluated the cut-off of 0.5, 0.4, 0.3, 0.2 and 0.1 according to the prior art.

### Result

The motif search was performed against the UniProt protein database (datasets “Reviewed (Swiss-Prot)” and “Isoform sequences”, release version 2020_06¹). ¹ https://www.uniprot.org/downloads #uniprotkblink, accessed on 2021-02-10

The full-scan detects the off-target at all cut-offs below 0.5. At the optimal cut-off of 0.4 predicts 4 off-targets in total.

TABLE 12

| Cut-off | #Predicted off- | Off-target |
|---|---|---|
| | targets | found? |
| 0.5 | 3 | No |
| 0.4 | 4 | Yes |
| 0.3 | 11 | Yes |
| 0.2 | 157 | Yes |
| 0.1 | 1,393 | Yes |

3.3 Comparison with a Partial-Scan as Described in the Art

The use of a partial-scan to identify off-targets had been described by Karapetyan et al and in section 2.3 above. We evaluated the cut-offs 0.905 and 0.85 that are proposed in the publication.

### Results

The search was performed against the 10-mer-digested UniProt protein database (datasets “Reviewed (Swiss-Prot)” and “Isoform sequences”, release version 2020_06¹). The 10-mer were pre-filtered with NetMHC 3.0 (predicted HLA A*03:01 affinity≤500 nM). ¹ https://www.uniprot.org/downloads #uniprotkblink, accessed on 2021-02-10

The off-target is detected at both cut-offs with 9 (0.905) and 88 (0.85) total predicted off-targets.

As described in 2.3, we also looked at cut-offs lower than those proposed in Front. Immunol., 22 Oct. 2019.

TABLE 13

| | #Predicted off- | Off-target |
|---|---|---|
| Cut-off | targets | found? |
| 0.905 | 9 | Yes |
| 0.850 | 88 | Yes |
| 0.800 | 329 | Yes |
| 0.750 | 833 | Yes |
| 0.700 | 3,341 | Yes |
| 0.650 | 7,482 | Yes |

### Example 4: Using Amino Acid Similarity Measurements to Provide a Position-Specific Scoring Matrix (PSSM)

The method for the identification of alternative target peptides (i.e. the second aspect of the invention) used in the above examples can also be used without any substitution analysis partial- or full-scan. Such a method could for example be based on amino acid similarity measures. The following examples uses binding affinity data from the peptide-MHC binding energy covariance (PMBEC; see Kim et al; BMV Bioinformatics 2009; 10:394). This amino acid similarity matrix is directly derived from the binding affinity data of combinatorial peptide mixtures. One prominent feature of the PMBEC matrix is that it disfavors substitution of residues with opposite charges. Of note the following example could be used with ANY amino acid similarity measures. Another well-known example that could be used is BLOSUM62.

### 4.1 Use of the PMBEC Matrix to Replace Mutational Scan

The PMBEC matrix provides an amino acid similarity value for the replacement of every proteinogenic amino acid with any other proteinogenic amino acid. Thus for any amino acid in the target peptide a list of values for the alternative amino acids at this position can be determined from the PMBEC matrix resulting in a position-specific scoring matrix (PSSM).

For a target peptide with 9 amino acids a matrix with 9×20 cells is created. The values derived from PMBEC are transformed by raising them to the power of 2 in order to avoid negative values. This replaces the data derived from the mutational scan in the previous examples.

### 4.2 UniProt Protein Database Digestion

To determine the list of peptides that could theoretically be presented by the target HLA, we downloaded the UniProt protein database (datasets “Reviewed (Swiss-Prot)” and “Isoform sequences”, release version 2020_06²) in FASTA format which contains 604,375 protein sequences. We merged the two datasets and filtered it to include only proteins of Homo sapiens (tax ID 9606) which resulted in a total of 42,383 human protein sequences. All proteins in the merged dataset were in-silico digested into 9-mers (amino acids of length 9). For the in-silico digest, we included overlapping 9-mers. For example, the protein sequence “MTMDKSELVQK” (SEQ ID NO: 26) was digested into the 9-mers “MTMDKSELV” (SEQ ID NO: 27), “TMDKSELVQ” (SEQ ID NO: 28), and “MDKSELVQK” (SEQ ID NO: 29). The resulting 9-mers were sorted and uniquified. This resulted in 10,797,420 unique 9-mers. ² https://www.uniprot.org/downloads #uniprotkblink, accessed on 2021-02-10

### 4.3 Binding Score Calculation

For each peptide ρ in the digested UniProt peptide database, we calculated the binding score S_{ρ} as

$${S}_{p} = {\sum }_{i = 1}^{9} {log}_{2} {B}_{{p}_{i} , i}$$

where ρᵢ is the amino acid at position i in peptide ρ and B_{ρ}_{ᵢ}_{,i} is the binding value determined by the PSSM for the mutational variant that has amino acid ρᵢ at positioni.

### 4.4 Alternative Target Peptide Selection

To determine if a peptide ρ is a predicted off-target, the binding score S_{ρ} is compared with a cut-off. We evaluated different numbers of predicted alternative targets between 10 and 200 to select alternative targets for experimental binding analysis.

To determine the number of true alternative target peptides that would be identified by measuring the binding value of a fixed number of potential alternative target peptides, we sorted the peptides in the UniProt database by their binding score calculated in 4.3 and determined the number of true alternative target peptides (see Example 1, 1.4).

### Results

The table below shows the results obtained with the PSSM based on PMBEC. The search for potential off-targets was done in the 9-mer-digested UniProt protein database (only human proteins, with isoforms).

TABLE 14

| #Potential | | | |
|---|---|---|---|
| alternative | | | |
| target peptides | #True positives | Recall | Precision |
| 10 | 1 | 0.07 | 0.10 |
| 20 | 1 | 0.07 | 0.05 |
| 50 | 2 | 0.13 | 0.04 |
| 100 | 2 | 0.13 | 0.02 |
| 200 | 2 | 0.13 | 0.01 |

The table below shows the results obtained with the PSSM based on PMBEC. The search for potential off-targets was done in the XPRESIDENT® ligandome database (see Zhang, et al.; Nat Commun 9, 3919 (2018)).

TABLE 15

| #Potential | | | |
|---|---|---|---|
| alternative | | | |
| target peptides | #True positives | Recall | Precision |
| 10 | 1 | 0.07 | 0.10 |
| 20 | 2 | 0.13 | 0.10 |
| 50 | 3 | 0.20 | 0.06 |
| 100 | 3 | 0.20 | 0.03 |
| 200 | 4 | 0.27 | 0.02 |

### Example 5: Using Amino Acid Similarity Measurements in Combination with a Single-Scan to Provide a Position-Specific Scoring Matrix (PSSM)

The use of an amino acid similarity matrix such as PMBEC of example 4 can also be combined with a mutational scan, in particular a single-scan that only uses a single amino acid as an alternative on each position. In the present examples a single-scan was used that replaces each amino acid of the target peptide with an alanine and measures binding affinity of each target peptide variant having one amino acid replaced with alanine.

5.1 Combination of the PMBEC Matrix with the Single-Scan

The results from the single-scan are then used to identify relevant positions in the target peptide. Therefore each amino acid position being replaced by alanine that results in a target peptide variant having less than 50% binding affinity than the initial target peptide is considered as a relevant position. For the relevant positions a PSSM as described in 4.1 above is created, whereas the non-relevant position are assigned a value of zero. The values in the PSSM are transformed by raising them to the power of 2 in order to avoid negative values. This replaces the data derived from the mutational scan or the PMBEC matrix alone in the previous examples.

### 5.2 Alternative Target Peptide Selection

The further determination of the alternative target peptide is identical as described above in example 4 (steps 4.2, 4.3 and 4.4).

### Results

The table below shows the results obtained with the PSSM based on PMBEC and a single-scan. The search for potential off-targets was done in the 9-mer-digested UniProt protein database (only human proteins, with isoforms).

TABLE 16

| #Potential | | | |
|---|---|---|---|
| alternative | | | |
| target peptides | #True positives | Recall | Precision |
| 10 | 1 | 0.07 | 0.10 |
| 20 | 1 | 0.07 | 0.05 |
| 50 | 1 | 0.07 | 0.02 |
| 100 | 1 | 0.07 | 0.01 |
| 200 | 3 | 0.20 | 0.02 |

The table below shows the results obtained with the PSSM based on PMBEC and a single-scan. The search for potential off-targets was done in the XPRESIDENT® ligandome database (see Zhang, et al.; Nat Commun 9, 3919 (2018)).

TABLE 17

| #Potential | | | |
|---|---|---|---|
| alternative | | | |
| target peptides | #True positives | Recall | Precision |
| 10 | 3 | 0.20 | 0.30 |
| 20 | 5 | 0.33 | 0.25 |
| 50 | 6 | 0.40 | 0.12 |
| 100 | 7 | 0.47 | 0.07 |
| 200 | 10 | 0.67 | 0.05 |

The table below contains the area on the precision-recall curves (PRC-AUC) for all methods shown in FIGS. 7, 8, and 9.

TABLE 18

| | | UniProt + | |
|---|---|---|---|
| | XPRESIDENT | NetMHC | UniProt |
| single+BLOSUM | 0.127 | 0.062 | 0.040 |
| single+PMBEC | 0.184 | 0.078 | 0.043 |
| BLOSUM | 0.097 | 0.081 | 0.071 |
| partial-scan | 0.345 | 0.153 | 0.044 |
| partial-scan (logsum) | 0.369 | 0.194 | 0.045 |
| PMBEC | 0.089 | 0.080 | 0.071 |
| XPRES-Scan (LOD, | 0.676 | 0.306 | 0.194 |
| 0.5%) | | | |
| XPRES-Scan (LOD, | 0.602 | 0.350 | 0.267 |
| 1%) | | | |
| XPRES-Scan | 0.571 | 0.284 | 0.177 |
| (median, 0.5%) | | | |
| XPRES-Scan | 0.506 | 0.267 | 0.203 |
| (median, 1%) | | | |
| XPRES-Scan (min, | 0.669 | 0.305 | 0.191 |
| 0.5%) | | | |
| XPRES-Scan (min, | 0.592 | 0.347 | 0.262 |
| 1%) | | | |
| full-scan | 0.231 | 0.149 | 0.074 |
| full-scan (logsum) | 0.547 | 0.265 | 0.030 |

1. A method for the identification of alternative target peptides of a binding moiety comprising the steps of:
a) providing a target peptide of the binding moiety;
b) generation of a replacement matrix for the target peptide, wherein the replacement matrix provides a selection of alternative amino acids for amino acids of the target peptide,
with the proviso that
the replacement matrix contains less alternative amino acids than the number of all possible alternative amino acids and
the selection of alternative amino acids is not identical in all selections;
c) generating peptide variants of the target peptide according to the replacement matrix of step b), wherein each peptide variant comprises only one alternative amino acid as compared to the target peptide; determining at least one binding parameter of the peptide variants to the binding moiety;
d) providing a list of potential alternative target peptides of the binding moiety comprising the following steps:
i) calculating a position-specific binding value for the alternative amino acid present in the peptide variant of step c), based on the at least one binding parameter of the peptide variant determined in step c),
ii) providing a list of amino acid sequences having a specified length and being comprised in a protein database, optionally a proteome or ligandome database,
iii) assigning an alternative target peptide value to each amino acid sequence of step ii) based on the position-specific binding values for each alternative amino acid present in the amino acid sequence;
iv) selecting the potential alternative target peptides from the list of amino acid sequences of step ii) based on the binding peptide values assigned in step iii);
e) determining at least one binding parameter of the potential alternative target peptides to the binding moiety, wherein alternative target peptides are identified based on at least one binding parameter.

2. A method for the identification of alternative target peptides of a binding moiety based on a substitution analysis of a target peptide of said binding moiety, comprising the following steps:
I) determining a position-specific binding value for each alternative amino acid in the mutational scan of the target peptide, based on at least one binding parameter determined in the substitution analysis;
II) providing a list of amino acid sequences having a specified length of the target peptide and being comprised in a protein database, optionally a proteome or ligandome database;
III) assigning an alternative target peptide value to each amino acid sequence of step II) based on the position-specific binding values of each alternative amino acid of step I) present in the amino acid sequence;
IV) selecting the potential alternative target peptides from the list of amino acid sequences of step II) based on the alternative target peptide values assigned in step III);
V) determining at least one binding parameter of the potential alternative target peptides to the binding moiety, wherein alternative target peptides are identified based on at least one binding parameter.

3. The method according to claim 2, wherein the substitution analysis comprises the following steps:
a) providing the target peptide of the binding moiety;
b) generation of a position-specific scoring matrix (PSSM) for the target peptide, wherein the PSSM provides a selection of amino acids for each position of the target peptide, optionally wherein the selection of amino acids is any proteinogeic amino acid;
c) assigning a value based on an amino acid similarity measure to each cell of the PSSM.

4. The method according to claim 1, wherein
(a) the binding moiety is selected from:
a T-cell receptor (TCR), TCR-derivative or fragment thereof, or
an antibody, derivative or antigen binding fragment thereof,
optionally the binding moiety is selected from a TCR, TCR-derivative or fragment thereof; and/or
(b) the target peptide is presented by a Major Histocompatibility Complex (MHC), optionally by MHC I.

5. The method according to claim 1, wherein the replacement matrix of step b) is generated by determining for each amino acid of the target peptide the frequency of occurrence for every alternative amino acid in a suitable ligandome database, wherein each alternative amino acid with a frequency of occurrence of at least 2%, at least 1.5%, at least 1%, at least 0.5% is included in the selection of alternative amino acids for each amino acid of the target peptide.

6. The method according to claim 1, wherein the replacement matrix of step b) provides a selection of alternative amino acids for at least 5, at least 6, at least 7, at least 8, at least 9, each amino acid(s) of the target peptide.

7. The method according to claim 1,
wherein the at least one binding parameter
in step c) and/or step e); or
step I) and/or step V)
are selected from:
binding affinity of the binding moiety to the peptide variant;
association rate of the binding moiety to the peptide variant;
dissociation rate of the binding moiety to the peptide variant;
release of cytokines, optionally interferon γ, from a host cell expressing the binding moiety in response to binding the peptide variant;
surface activation markers on a host cell expressing the binding moiety in response to binding the peptide variant;
proliferation of a T cell in response to binding the peptide variant;
cytotoxicity of a T cell in response to binding the peptide variant.

8. The method according to claim 1, wherein the proteome database of step d), ii) or step II) is modified, wherein all proteins of the proteome database are in silico digested into all possible peptides of the same length as the target peptide.

9. The method according to claim 1, wherein the position-specific binding value for each alternative amino acid of step d) i) or step I), is expressed in relation to the binding value of the amino acid of the target peptide.

10. The method according to claim 1, wherein the alternative target peptide value of step d) iii) or step III) is additionally based on:
a binding value for each amino acid of the target peptide that is present in the amino acid sequence; and/or
a position-specific binding value of each possible alternative amino acid not being included in the selection of alternative amino acids of step b) or the mutational scan of the target peptide, wherein the position-specific binding value is determined by the position-specific binding value of the alternative amino acids at the same position, optionally selected from a median, a mean, a minimum, and a maximum, optionally a median or mean, optionally a median.

11. The method according to claim 1, wherein the alternative target peptide value of step d) iii) or step III) is determined by calculating a sum of the logarithmic binding values of each amino acid present in the amino acid sequence of step ii) or step II).

12. The method according to claim 1, wherein the selection of potential alternative target peptides in step d) iv) or step IV) is determined by:
applying a cut-off value to the alternative target peptide values; or
ranking the alternative target peptide value of step d) iii) or step III) and selecting the 10, 20, 30, 40, 50, 60, 70, 80, 80 or 100, optionally 10, 60 or 100, highest ranking potential alternative target peptides.

13. The method according to claim 1, wherein the alternative amino acids of step b) or in the mutational scan are selected from alanine, glycine and proteinogenic amino acids, optionally from proteinogenic amino acids.

14. The method according to claim 1, wherein the proteome database of step d) ii) or step II) is modified by including only peptides identified in an in silico prediction that the peptides are MHC presented peptides, optionally MHC-I presented peptides.

15. The method according to claim 1, wherein the ligandome database of step d) ii) or step II); or, if used, the suitable ligandome database of step b):
is a ligandome database of MHC presented peptides, optionally of MHC 1 presented peptides;
comprises at least 200, 300, 400, 500, 600, 700, 800, 900, 1000 or more peptides; and/or
comprises only peptides validated by mass spectrometry.

16. The method according to claim 2, wherein
(a) the binding moiety is selected from:
a T-cell receptor (TCR), TCR-derivative or fragment thereof, or
an antibody, derivative or antigen binding fragment thereof,
optionally the binding moiety is selected from a TCR, TCR-derivative or fragment thereof; and/or
(b) the target peptide is presented by a Major Histocompatibility Complex (MHC), optionally by MHC I.

17. The method according to claim 2, wherein the replacement matrix of step b) is generated by determining for each amino acid of the target peptide the frequency of occurrence for every alternative amino acid in a suitable ligandome database, wherein each alternative amino acid with a frequency of occurrence of at least 2%, at least 1.5%, at least 1%, at least 0.5% is included in the selection of alternative amino acids for each amino acid of the target peptide.

18. The method according to claim 2, wherein the replacement matrix of step b) provides a selection of alternative amino acids for at least 5, at least 6, at least 7, at least 8, at least 9, each amino acid(s) of the target peptide.

19. The method according to claim 2, wherein the at least one binding parameter
in step c) and/or step e); or
step I) and/or step V)
are selected from:
binding affinity of the binding moiety to the peptide variant;
association rate of the binding moiety to the peptide variant;
dissociation rate of the binding moiety to the peptide variant;
release of cytokines, optionally interferon γ, from a host cell expressing the binding moiety in response to binding the peptide variant;
surface activation markers on a host cell expressing the binding moiety in response to binding the peptide variant;
proliferation of a T cell in response to binding the peptide variant;
cytotoxicity of a T cell in response to binding the peptide variant.

20. The method according to claim 2, wherein the proteome database of step d), ii) or step II) is modified, wherein all proteins of the proteome database are in silico digested into all possible peptides of the same length as the target peptide.


SEQUENCE LISTING (32 sequences)

SEQ ID NO: 1 - protein, 461 aa, synthetic construct
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 2 - protein, 117 aa, synthetic construct
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQN

SEQ ID NO: 3 - protein, 6 aa, synthetic construct
DRGSQS

SEQ ID NO: 4 - protein, 6 aa, synthetic construct
IYQEGD

SEQ ID NO: 5 - protein, 15 aa, synthetic construct
CAAVIDNDQGGILTF

SEQ ID NO: 6 - protein, 26 aa, synthetic construct
QKEVEQNSGPLSVPEGAIASLNCTYS

SEQ ID NO: 7 - protein, 17 aa, synthetic construct
FFWYRQYSGKSPELIMS

SEQ ID NO: 8 - protein, 33 aa, synthetic construct
KEDGRFTAQLNKASQYVSLLIRDSQPSDSATYL

SEQ ID NO: 9 - protein, 14 aa, synthetic construct
GTGTRLTIIPNIQN

SEQ ID NO: 10 - protein, 8 aa, synthetic construct
GGGSGGGG

SEQ ID NO: 11 - protein, 107 aa, synthetic construct
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIK

SEQ ID NO: 12 - protein, 229 aa, synthetic construct
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 13 - protein, 477 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 14 - protein, 118 aa, synthetic construct
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 15 - protein, 5 aa, synthetic construct
PGHRA

SEQ ID NO: 16 - protein, 6 aa, synthetic construct
YVHGEE

SEQ ID NO: 17 - protein, 14 aa, synthetic construct
CASSPWDSPNVQYF

SEQ ID NO: 18 - protein, 26 aa, synthetic construct
KAGVTQTPRYLIKTRGQQVTLSCSPI

SEQ ID NO: 19 - protein, 17 aa, synthetic construct
VSWYQQTPGQGLQFLFE

SEQ ID NO: 20 - protein, 36 aa, synthetic construct
RNKGNFPGRFSGRQFSNSSSEMNISNLELGDSALYL

SEQ ID NO: 21 - protein, 14 aa, synthetic construct
GPGTRLTVTEDLKN

SEQ ID NO: 22 - protein, 8 aa, synthetic construct
GGGSGGGG

SEQ ID NO: 23 - protein, 122 aa, synthetic construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSS

SEQ ID NO: 24 - protein, 229 aa, synthetic construct
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 25 - protein, 10 aa, synthetic construct
IIVGAIGVGK

SEQ ID NO: 26 - protein, 11 aa, synthetic construct
MTMDKSELVQK

SEQ ID NO: 27 - protein, 9 aa, synthetic construct
MTMDKSELV

SEQ ID NO: 28 - protein, 9 aa, synthetic construct
TMDKSELVQ

SEQ ID NO: 29 - protein, 9 aa, synthetic construct
MDKSELVQK

SEQ ID NO: 30 - protein, 10 aa, synthetic construct
VVVGAVGVGK

SEQ ID NO: 31 - protein, 9 aa, synthetic construct
FMNPHLISV

SEQ ID NO: 32 - protein, 9 aa, synthetic construct
YLLPAIVHI